SEQUENCE LISTING (19 sequences)

SEQ ID NO: 1 - protein, 462 aa, Oryza sativa
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 2 - protein, 462 aa, Oryza sativa
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGIAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 3 - protein, 442 aa, Solanum lycopersicum
MATNLRVLMFPWLAYGHISPFLNIAKQLADRGFLIYLCSTRINLESIIKKIPEKYADSIHLIELQLPELPELPPHYHTTNGLPPHLNPTLHKALKMSKPNFSRILQNLKPDLLIYDVLQPWAEHVANEQNIPAGKLLTSCAAVFSYFFSFRKNPGVEFPFPAIHLPEVEKVKIREILAKEPEEGGRLDEGNKQMMLMCTSRTIEAKYIDYCTELCNWKVVPVGPPFQDLITNDADNKELIDWLGTKHENSTVFVSFGSEYFLSKEDMEEVAFALELSNVNFIWVARFPKGEERNLEDALPKGFLERIGERGRVLDKFAPQPRILNHPSTGGFISHCGWNSAMESIDFGVPIIAMPIHNDQPINAKLMVELGVAVEIVRDDDGKIHRGEIAETLKSVVTGETGEILRAKVREISKNLKSIRDEEMDAVAEELIQLCRNSNKSK

SEQ ID NO: 4 - protein, 454 aa, Lycium barbarum
MGTEVTVHKNTLRVLMFPWLAYGHISPFLNVAKKLVDRGFLIYLCSTAINLKSTIKKIPEKYSDSIQLIELHLPELPELPPHYHTTNGLPPHLNHTLQKALKMSKPNFSKILQNLKPDLVIYDLLQQWAEGVANEQNIPAVKLLTSGAAVLSYFFNLVKKPGVEFPFPAIYLRKNELEKMSELLAQSAKDKEPDGVDPFADGNMQVMLMSTSRIIEAKYIDYFSGLSNWKVVPVGPPVQDPIADDADEMELIDWLGKKDENSTVFVSFGSEYFLSKEDREEIAFGLELSNVNFIWVARFPKGEEQNLEDALPKGFLERIGDRGRVLDKFAPQPRILNHPSTGGFISHCGWNSVMESVDFGVPIIAMPIHLDQPMNARLIVELGVAVEIVRDDYGKIHREEIAEILKDVIAGKSGENLKAKMRDISKNLKSIRDEEMDTAAEELIQLCKNSPKLK

SEQ ID NO: 5 - protein, 9 aa, Oryza sativa
WLAFGHLLP

SEQ ID NO: 6 - protein, 11 aa, Oryza sativa
NDVPHDRPDMV

SEQ ID NO: 7 - protein, 7 aa, Oryza sativa
LLGSAHM

SEQ ID NO: 8 - protein, 16 aa, Oryza sativa
RMKLIRTKGSSGMSLA

SEQ ID NO: 9 - protein, 9 aa, Oryza sativa
YVALGSEVP

SEQ ID NO: 10 - protein, 9 aa, Oryza sativa
RWVPQMSIL

SEQ ID NO: 11 - protein, 12 aa, Oryza sativa
FLTHCGWNSTIE

SEQ ID NO: 12 - protein, 8 aa, Oryza sativa
IFGDQGPN

SEQ ID NO: 13 - protein, 463 aa, Hordeum vulgare
MDGDGNSSSSSSPLHVVICPWLALGHLLPCLDIAERLASRGHRVSFVSTPRNIARLPPLRPAVAPLVEFVALPLPHVDGLPEGAESTNDVPYDKFELHRKAFDGLAAPFSEFLRAACAEGAGSRPDWLIVDTFHHWAAAAAVENKVPCVMLLLGAATVIAGFARGVSEHAAAAVGKERPAAEAPSFETERRKLMTTQNASGMTVAERYFLTLMRSDLVAIRSCAEWEPESVAALTTLAGKPVVPLGLLPPSPEGGRGVSKEDAAVRWLDAQPAKSVVYVALGSEVPLRAEQVHELALGLELSGARFLWALRKPTDAPDAAVLPPGFEERTRGRGLVVTGWVPQIGVLAHGAVAAFLTHCGWNSTIEGLLFGHPLIMLPISSDQGPNARLMEGRKVGMQVPRDESDGSFRREDVAATVRAVAVEEDGRRVFTANAKKMQEIVADGACHERCIDGFIQQLRSYKA

SEQ ID NO: 14 - protein, 436 aa, Setaria italica
MDASDSSPLHIVIFPWLAFGHMLASLELAERLAARGHRVSFVSTPRNISRLRPVPPALAPLIDFVALPLPRVDGLPDGAEATSDIPPGKTELHLKALDGLAAPFAAFLDAACADGSTNKVDWLFLDNFQYWAAAAAADHKIPCALNLTFAASTSAEYGVPRVEPPVDGSTASILQRFVLTLEKCQFVIQRACFELEPEPLPLLSDIFGKPVIPYGLVPPCPPAEGHKREHGNAALSWLDKQQPESVLFIALGSEPPVTVEQLHEIALGLELAGTTFLWALKKPNGLLLEADGDILPPGFEERTRDRGLVAMGWVPQPIILAHSSVGAFLTHGGWASTIEGVMSGHPMLFLTFLDEQRINAQLIERKKAGLRVPRREKDGSYDRQGIAGAIRAVMCEEESKSVFAANAKKMQEIVSDRNCQEKYIDELIQRLGSFEK

SEQ ID NO: 15 - protein, 473 aa, Stevia rebaudiana
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGMVLKGSDCLLSKCYHEFGTQWLPLLETLHQVPVVPVGLLPPEIPGDEKDETWVSIKKWLDGKQKGSVVYVALGSEALVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 16 - protein, 442 aa, Panax ginseng
MDNQNGRISIALLPFLAHGHISPFFELAKQLAKRNCNVFLCSTPINLSSIKDKDSSASIKLVELHLPSSPDLPPHYHTTNGLPSHLMLPLRNAFETAGPTFSEILKTLNPDLLIYDFNPSWAPEIASSHNIPAVYFLTTAAASSSIGLHAFKNPGEKYPFPDFYDNSNITPEPPSADNMKLLHDFIACFERSCDIILIKSFRELEGKYIDLLSTLSDKTLVPVGPLVQDPMGHNEDPKTEQIINWLDKRAESTVVFVCFGSEYFLSNEELEEVAIGLEISTVNFIWAVRLIEGEKKGILPEGFVQRVGDRGLVVEGWAPQARILGHSSTGGFVSHCGWSSIAESMKFGVPVIAMARHLDQPLNGKLAAEVGVGMEVVRDENGKYKREGIAEVIRKVVVEKSGEVIRRKARELSEKMKEKGEQEIDRALEELVQICKKKKDEQ

SEQ ID NO: 17 - protein, 446 aa, Solanum lycopersicum
MSTTLKVLMFPFLAYGHISPYLNVAKKLADRGFLIYLCSTPINLKSTINKIPEKYADSIQLIELHLPELPELPPHYHTTNGLPPNLNHILRRALKMSKPNFSKIMQNLKPDLLIYDILQQWAEDVATELNIPAVKLLTSGVAVFSYFFNLTKKPEVEFPYPAIYLRKIELVRWCETLSKHNKEGEEHDDGLAYGNMQIMLMSTSKILEAKYIDYCIELTNWKVVPVGSLVQDSITNDAADDDMELIDWLGTKDENSTVFVSFGSEYFLSKEDVEEVAFGLELSNVNFIWVVRFPKGEEKNLEDVLPKGFFERIGERGRVLDKFAPQPRILNHPSTGGFISHCGWNSAMESIDFGVPIVAMPMQLDQPMNARLIVELGVAVEIVRDDDGKIYRGEIAETLKGVITGEIGEILRAKVRDISKNLKAIKDEEMDVAAQELIQLCRNSNK

SEQ ID NO: 18 - protein, 470 aa, Solanum lycopersicum
MSPKLHKELFFHSLYKKTRSNHTMATLKVLMFPFLAYGHISPYLNVAKKLADRGFLIYFCSTPINLKSTIEKIPEKYADSIHLIELHLPELPQLPPHYHTTNGLPPNLNQVLQKALKMSKPNFSKILQNLKPDLVIYDILQRWAKHVANEQNIPAVKLLTSGAAVFSYFFNVLKKPGVEFPFPGIYLRKIEQVRLSEMMSKSDKEKELEDDDDDDDLLVDGNMQIMLMSTSRTIEAKYIDFCTALTNWKVVPVGPPVQDLITNDVDDMELIDWLGTKDENSTVFVSFGSEYFLSKEDMEEVAFALELSNVNFIWVARFPKGEERNLEDALPKGFLERIGERGRVLDKFAPQPRILNHPSTGGFISHCGWNSAMESIDFGVPIIAMPMHLDQPMNARLIVELGVAVEIVRDDDGKIHRGEIAETLKGVITGKTGEKLRAKVRDISKNLKTIRDEEMDAAAEELIQLCRNGN

SEQ ID NO: 19 - protein, 442 aa, Solanum lycopersicum; identical to SEQ ID NO: 3

(19) United States
(12) Patent Application Publication
(10) Pub. No.: US 2024/0182941 A1
(43) Pub. Date: Jun. 6, 2024

(54) XYLOSYLATED STEVIOL GLYCOSIDES AND ENZYMATIC METHODS FOR PRODUCTION

(71) Applicant: CARGILL, INCORPORATED, Wayzata, MN (US)

(72) Inventors: Erin EVANS, Robbinsdale, MN (US); Daniel Scott GASPARD, Victoria, MN (US); Panagiota KYRIAKOU, Minneapolis, MN (US); Erin Kathleen MARASCO, Excelsior, MN (US); Guo-Hua ZHENG, Centerville, OH (US)

(73) Assignee: CARGILL, INCORPORATED, Wayzata, MN (US)

(21) Appl. No.: 18/548,730

(22) PCT Filed: Mar. 1, 2022
(86) PCT No.: PCT/US2022/070906

§ 371 Date: Sep. 1, 2023

Related U.S. Application Data
(60) Provisional application No. 63/155,229, filed on Mar. 1, 2021.

(51) Int. Cl. C12P 19/56; C12N 9/10; C12P 19/18

(52) U.S. Cl. CPC C12P 19/56; C12N 9/1077; C12P 19/18; C12Y 204/02

(57) ABSTRACT

Disclosed are methods and compositions for producing xylosylated steviol glycosides that include a xylose residue, or an oligosaccharide moiety including a xylose residue, attached to the 19 carbon of the steviol base. A glycosyltransferase having UDP-xylose:19-steviol xylosyltransferase activity (such as one of SEQ ID NO:1 or a homolog or variant thereof) forms the xylosylated steviol glycoside using a steviol glycoside acceptor having a glucose residue attached to the 19 carbon of the steviol base. Glycosyltransferase variants with increased activity are also described.

### CROSS-REFERENCE TO RELATED APPLICATIONS

This application claims the benefit of U.S. Provisional Patent Application No. 63/155,229, filed Mar. 1, 2021, which is incorporated by reference herein in its entirety.

### REFERENCE TO A SEQUENCE LISTING SUBMITTED VIA EFS-WEB

The content of the ASCII text file of the sequence listing named “PT_923_WO_ST25.txt” which is 43.7 kb in size created on Mar. 1, 2022 and electronically submitted vis EFS-Web herewith the application is incorporated by reference in its entirety.

### FIELD

The present disclosure relates to xylosylated steviol glycosides and enzymatic methods for their production.

### BACKGROUND

Sugars, such as sucrose, fructose and glucose, are utilized to provide a pleasant taste to beverages, foods, pharmaceuticals, and oral hygienic/cosmetic products. Sucrose, in particular, imparts a taste preferred by consumers. Although sucrose provides superior sweetness characteristics, it is caloric. Non-caloric or lower caloric sweeteners have been introduced to satisfy consumer demand, and there is desire for these types of sweeteners that have favorable taste characteristics.

Stevia is a genus of about 240 species of herbs and shrubs in the sunflower family (Asteraceae), native to subtropical and tropical regions from western North America to South America. The species Stevia rebaudiana, commonly known as sweetleaf, sweet leaf, sugarleaf, or simply stevia, is widely grown for its sweet leaves. Stevia-based sweeteners may be obtained by extracting one or more sweet compounds from the leaves. Many of these compounds are steviol glycosides, which are glycosides of steviol, a diterpene compound. These diterpene glycosides are about 150 to 450 times sweeter than sugar.

Examples of steviol glycosides are described in WO 2013/096420 (see, e.g., listing in FIG. 1); and in Ohta et al., (2010) “Characterization of Novel Steviol Glycosides from Leaves of Stevia rebaudiana Morita,” J. Appl. Glycosi., 57:199-209 (See, e.g., Table 4 at p. 204). Structurally, the diterpene glycosides are characterized by a single base, steviol, and differ by the presence of carbohydrate residues at positions C13 and C19, as presented in FIGS. 2a-2k. See also PCT Patent Publication WO 20013/096420.

The most abundant steviol glucosides in S. rebaudiana are 1,2-stevioside, rebaudioside A (Reb A) and rebaudioside C (Reb C) constituting 5-10, 2-4 and 1-2% of the leaf dry weight, respectively (Chatsudthipong, V., and Muanprasat, C. (2009) Stevioside and related compounds: therapeutic benefits beyond sweetness. Pharmacol Ther.; 121:41-54.) Reb A is 250-300 times more sweet than sucrose but possesses a bitter lingering aftertaste (Prakash, I., et al. (2014) Development of next generation stevia sweetener: rebaudioside M. Foods 3:162-75). Reb C is only 30 times sweeter than glucose (differentiated by a rhamnose moiety attached to the glucose moiety at the C13 position (DuBois, G. E., and Stephenson, R. A. (1985) Diterpenoid sweeteners. Synthesis and sensory evaluation of stevioside analogues with improved organoleptic properties. J. Med. Chem. 28:93-8.]. Rebaudioside D (Reb D) and Rebaudioside M (Reb M) have a sweetness potency up to 350 times that of sucrose, with less bitterness (Prakash). Reb M is characterized by a high sweetness intensity, a fast sweetness on-set, a clean taste and with greatly reduced licorice, bitter, sour and astringent aftertaste in comparison to Reb A and other steviol glucosides (Prakash). Reb D and Reb M are only present in the leaves of S. rebaudiana in minute quantities (approximately 0.4-1.5% w/w total dry weight in traditional plants from China).

### SUMMARY

The present disclosure provides enzymatic methods for producing xylosylated steviol glycosides. In aspects, xylosylation can be performed in a reaction composition, or can be performed in engineered cells. The present disclosure also provides engineered cells capable of producing xylosylated steviol glycosides, as well as compositions that include xylosylated steviol glycosides. Aspects of the disclosure are based on the experimental findings associated with the identification of glycosyltransferase polypeptides having UDP-xylose:19-steviol xylosyltransferase activity (including those of SEQ ID NO:1, SEQ ID NO:2, and SEQ ID NO:3) that can utilize activated xylose to provide steviol glycosides having a xylose residue, or an oligosaccharide moiety including a xylose residue, attached to the 19C position of the steviol base.

In some aspects the disclosure provides a method for forming a xylosylated steviol glycoside. The method includes forming a compound of Formula II from a compound of Formula I using a glycosyltransferase having UDP-xylose:19-steviol xylosyltransferase activity in vitro or in an engineered cell. Formula I is:

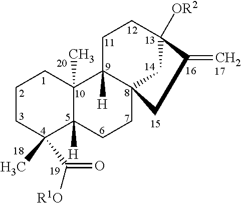

wherein R¹ comprises a glucose residue, and R² comprises one or more sugar residues or is a hydrogen. The glycosyltransferase transfers xylose from an activated xylose to the compound of Formula Ito form a compound of Formula II:

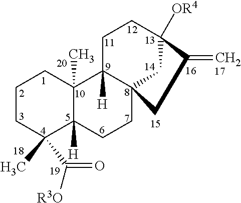

wherein R³ comprises one or more xylose residue(s) that are added by the glycosyltransferase, and R⁴ is the same as R² or comprises one or more additional sugar residue(s).

Glycosyltransferase polypeptides having UDP-xylose:19-steviol xylosyltransferase activity include those of SEQ ID NO:1, SEQ ID NO:2, and SEQ ID NO:3, variants thereof, and homologues thereof.

In the xylosylated product of Formula II, R³ includes a xylose residue, and R⁴ can the same chemistry as R² in the compound of Formula I, or R⁴ can include one or more additional sugar residues as compared to R². In aspects, R⁴ is an oligosaccharide moiety comprising a xylose residue, such as β-Glc-β-Xyl, which can be have a Glc2C→Xyl1C glycosidic linkage. In aspects, R⁴ can include one or more other saccharide residues, such as glucose or rhamnose. In yet other aspects, the compound of Formula I which becomes xylosylated at the 19 position is selected from stevioside, rebaudioside A (RebA), rebaudioside C (RebC), rebaudioside D (RebD), rebaudioside F, rebaudioside G, and dulcoside-A, with stevioside, RebA, and RebC being preferred substrates.

In some approaches, the method disclosed herein provides a compound of Formula II:

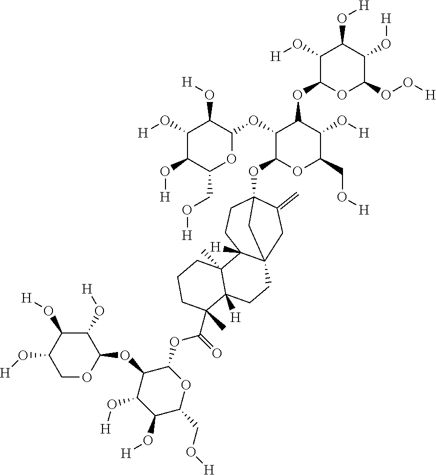

Chemically annotated SG-[13-β-Glc[(3-43 1)β-Glc](2→1)β-Glc(1-2)]-[19-β-Glc(2→1)β-Xyl and named rebaudioside DG (RebDG).

In aspects, the disclosure provides a method of forming the forming a xylosylated steviol glycoside, wherein the method uses a reaction composition. The reaction composition includes (i) the steviol glycoside compound of Formula I, (ii) an activated xylose; and (iii) a glycosyltransferase having UDP-xylose:19-steviol xylosyltransferase activity. A product composition is formed from the reaction composition wherein a xylosylated steviol glycoside compound of Formula II, such as RebDG, is formed by the glycosyltransferase transferring xylose from the activated xylose to the compound of Formula I.

In aspects, in the reaction composition the compound of Formula I can be present in an amount more than 50% (mol) and up to 100% (mol) of steviol glycoside acceptor therein. The reaction composition can include components such as non-activated sugar(s) and salt(s), a pH in a desired range, activated xylose, steviol glycoside acceptor, and a desired amount of polypeptide relative to the activated xylose to steviol glycoside acceptor to provide optimized reaction conditions for the transfer of xylose to the steviol glycoside acceptor. In some aspects activated xylose is used in molar excess to steviol glycoside acceptor. In the method, more than 2%, 5%, 7%, 10%, 20%, 30%, 40%, 50%, 60%, 65%, 75%, or more than 80% of the steviol glycoside compound of Formula I can be converted to a xylosylated steviol glycoside compound of Formula II, such as RebDG.

In other aspects, the disclosure provides a method for forming a xylosylated steviol glycoside using an engineered cell, wherein the engineered cells is capable of making a steviol glycoside compound of Formula I and where the cell expresses a glycosyltransferase having UDP-xylose:19-steviol xylosyltransferase activity, such as one of SEQ ID NO:1, SEQ ID NO:2, or SEQ ID NO:3, or a polypeptide having at least 50% identity to the glycosyltransferase polypeptide of any of SEQ ID NOs:1-3.

Activated xylose is provided in the engineered cell, such as by feeding activated xylose to the cell from an external source, or the engineered cell is capable of making activated xylose. The polypeptide transfers xylose from the activated xylose to the compound of Formula I to form a xylosylated steviol glycoside compound of Formula II. In some aspects the cell can be fed a steviol glycoside (precursor) compound which is used to make a steviol glycoside compound of Formula I which is used as the acceptor molecule. In some aspects, the cell is fed a steviol glycoside compound of Formula I, such as RebA, which can then be directly used as an acceptor for xylosylation to form a compound of Formula II, such as RebDG.

In other aspects, the disclosure provides an engineered cell that has a pathway for forming a steviol glycoside compound of Formula I. The cell is engineered to provide the steviol glycoside compound of Formula I, such as stevioside, RebA, or RebC, in an amount that is greater than an amount of steviol glycoside not of formula I that may be formed in the cell. The cell also expresses a glycosyltransferase having UDP-xylose:19-steviol xylosyltransferase activity, wherein the polypeptide is capable of transferring xylose from the activated xylose to the compound of Formula I to form a xylosylated steviol glycoside compound of Formula II.

In other aspects, the disclosure provides an ingestible or aqueous composition comprising mixture of steviol glycosides comprising a compound of Formula II, wherein the compound of Formula II, such as RebDG, is present in an amount greater than any other single steviol glycoside in the composition. In a further aspect, there are 2 or more compounds of Formula II in such an ingestible or aqueous composition and compounds of Formula II are present in a total amount greater than the total amount of other steviol glycosides in the composition. In another aspect, an ingestible or aqueous composition comprising a mixture of steviol glycosides, which optionally includes rebaudioside M, comprising a compound of Formula II present in an amount greater than rebaudioside M, if any rebaudioside M is present, and the compound of Formula II optionally comprises 1% (mol) or greater, e.g., 2%, 3%, or 5% (mol) or greater, of all steviol glycosides in the ingestible or aqueous composition.

The composition that includes the mixture of steviol glycosides can be obtained from a reaction composition or engineered cell that includes the glycosyltransferase having UDP-xylose:19-steviol xylosyltransferase activity.

In yet another aspect, the disclosure provides engineered glycosyltransferase variants having surprising high levels of UDP-xylose:19-steviol xylosyltransferase activity. The glycosyltransferase variants include a variant amino acid that changes a serine (wild type) to isoleucine (variant) residue at position 152 relative to SEQ ID NO:1. The amino acid change results in remarkable increases in xylosylation of a steviol glycoside molecule at the 19 position in the presence of activated xylose.

Accordingly, in another aspect, the disclosure provides a glycosyltransferase variant having UDP-xylose:19-steviol xylosyltransferase activity comprising a polypeptide having 50% or greater, 90% or greater, 95% or greater, or 98% or greater identity to SEQ ID NO:1 and the following amino acid: 1152. In some aspects, the glycosyltransferase variant is SEQ ID NO:2, which has a sequence with a single serine to isoleucine change at position 152 relative to SEQ ID NO:1.

### BRIEF DESCRIPTION OF THE DRAWINGS

FIG. 1 shows the chemical structure of a steviol base and a table of various steviol glycosides with chemical definitions of R¹ and R² groups.

FIG. 2 shows mevalonate (MVA) pathways for the conversion of acetyl-CoA to isopentenyl diphosphate (IPP) and dimethylallyl diphosphate (DMAPP) using eukaryotic and/or Archea enzymes.

FIG. 3 shows a non-mevalonate (MEP) pathway for the enzymatic conversion of glyceraldehyde-3-phosphate (G3P) and pyruvate (PYR) to IPP and DMAPP.

FIG. 4 shows an enzyme pathway for the conversion of IPP and farnesyl pyrophosphate (FPP) to steviol.

FIG. 5 is a graph showing average %SG acceptor conversion with UDP-xylose using an enzyme of SEQ ID NO:1 and UGT76G1 enzymes.

FIG. 6 is the amino acid sequence of SEQ ID NO:1 (AC133334).

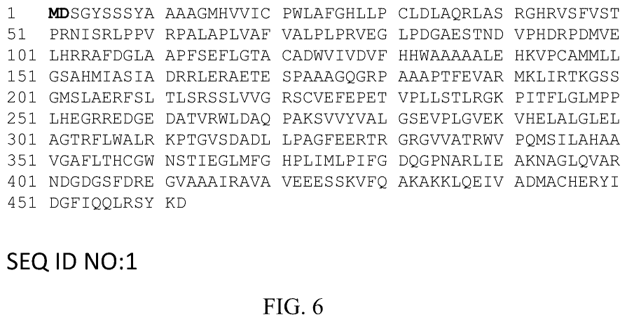

FIG. 7 is an amino acid sequence alignment of SEQ ID NO:1 with other glycosyltransferases or variants thereof.

FIG. 8A is a graph showing transfer of glucose and xylose from UDP-glucose and UDP-xylose to RebA over time in the presence of polypeptide of SEQ ID NO:1 and 1 mM activated sugars.

FIG. 8B is a graph showing transfer of glucose, xylose, and rhamnose from UDP-glucose, UDP-xylose, and UDP-rhamnose to RebA over time, in the presence of polypeptide of SEQ ID NO:1 and 2 mM activated sugars.

FIGS. 9A-C are graphs showing transfer glucose, xylose, and rhamnose from UDP-glucose, UDP-xylose, and UDP-rhamnose to the —19C(O))—β—Glc residue of Reb F, Reb G, and Dulcoside A, over time, in the presence of polypeptide of SEQ ID NO:1.

FIG. 10 is an amino acid sequence alignment of SEQ ID NO:1 with SEQ ID NOs:2-4.

### DETAILED DESCRIPTION

Aspects of the disclosure described herein are not intended to be exhaustive or to limit the claims to the precise forms disclosed in the following detailed description. Rather, a purpose of the aspects chosen and described is so that the appreciation and understanding by others skilled in the art of the principles and practices of the present disclosure can be facilitated.

Methods of the disclosure provide ways to form xylosylated steviol glycosides from steviol glycoside acceptor molecules using a glycosyltransferase having UDP-xylose:19-steviol xylosyltransferase activity, which in some aspects is a polypeptide having at least 50% identity to any of SEQ ID NOs:1-3, and an activated xylose molecule. It has been found that in the presence the glycosyltransferase polypeptide of SEQ ID NO:1 and activated xylose, steviol glycoside acceptor molecules are preferentially xylosylated on the 19 carbon position to form xylosylated steviol glycosides having a xylose residue attached to 19 carbon, or having one or more xylose residues present in in a oligosaccharide attached to 19 carbon.

The methods of the disclosure can be carried out using a reaction composition (e.g., an in vitro method not requiring an engineered cell) that includes a steviol glycoside acceptor, a glycosyltransferase having UDP-xylose:19-steviol xylosyltransferase activity, such as a glycosyltransferase polypeptide having at least 50% identity to any of SEQ ID NOs:1-3, and an activated xylose, such as UDP-xylose.

The methods of the disclosure can also be carried out using an engineered cell that expresses a glycosyltransferase having UDP-xylose:19-steviol xylosyltransferase activity, such as a glycosyltransferase polypeptide having at least 50% identity to any of SEQ ID NOs:1-3, and an activated xylose, such as UDP-xylose. The engineered cell can also have a pathway that makes a steviol glycoside compound that is acceptor molecule for the xylosylation at the 19 carbon position. In some aspects, the engineered cell can also have a pathway that makes activated xylose which can be utilized by the polypeptide to xylosylate the steviol glycoside compound. For example, the engineered cell can also include one or more of UDP-glucose dehydrogenase and/or UDP-glucuronic acid decarboxylase for the production of UDP-xylose. Alternatively, the engineered call can be fed activated xylose which can be taken up by the cell and used by the glycosyltransferase polypeptide. Alternatively, the engineered call can be fed a steviol glycoside molecule that is able to serve as an acceptor molecule for the xylosylation on the 19 C position, or serve as a precursor to the formation of the steviol glycoside acceptor molecule.

As used herein, the term “steviol glycoside(s)” refers to glycosides of steviol, that is, one or more sugar residues attached to a steviol base. Structurally, steviol glycosides have a central molecular moiety, which is a single steviol base, and sugar (glycopyronosyl) residues attached to the C13 and/or C19 atoms of the steviol base, according to the atom numbering on the base shown below. That is, one or more glycopyronosyl residues can be present in group(s) R¹ and/or R² in Formula I:

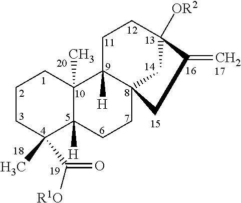

Glycopyronosyl residues that can be present in a steviol glycoside include those based on glucose, rhamnose, arabinose, and xylose. Other sugar residues such as fructose and deoxyglucose may be present in a steviol glycoside. If one or both of R¹ and/or R² have a single glycopyronosyl residue it can be referred to as a monosaccharide moiety or monosaccharide residue of the steviol glycoside. If one or both of R¹ and/or R² have two or more glycopyronosyl residues it can be referred to as an oligosaccharide moiety/residue of the steviol glycoside. An oligosaccharide moiety can consist of the same type of glycopyronosyl residues within the moiety (a homooligosaccharide moiety), or can consist of different types of glycopyronosyl residues within the moiety (a heterooligosaccharide moiety).

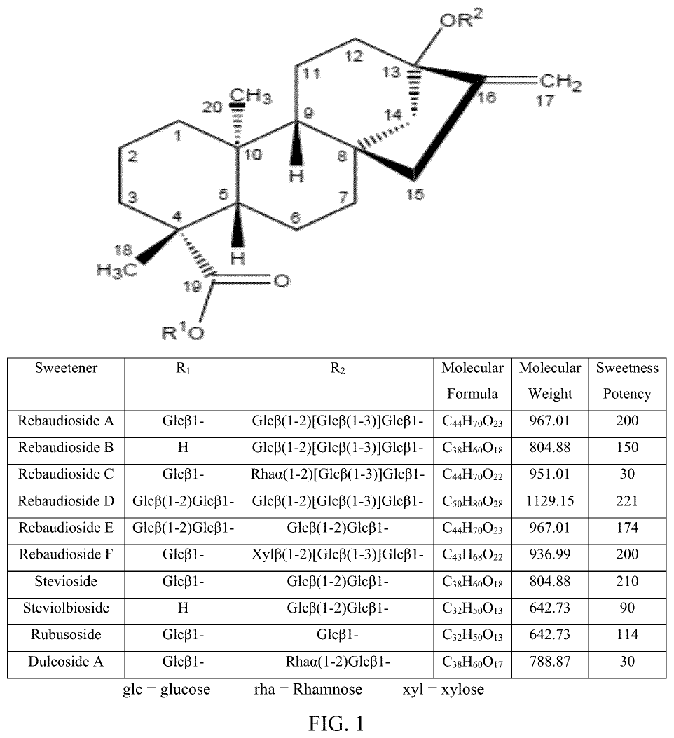

An oligosaccharide moiety can also be described in terms of the chemical linkage(s) between the glycopyronosyl residues in the oligosaccharide. For example, in an oligosaccharide moiety the linkages can be 1→2, 1→3, 1→4, or 1→6, alpha (α) or beta (β) glycosidic linkages, based on the numbering on the glycopyronosyl ring and the stereochemistry of the glycosidic linkage. In an alpha (α)glycosidic linkage the bond from the anomeric carbon to the oxygen of the glycosidic bond is oriented downwards from the glycopyronosyl ring, whereas in a beta (β) glycosidic linkages the bond is oriented upwards from the glycopyronosyl ring. Oligosaccahride moieties of the steviol glycoside can have a linear or branched configuration, with a branched configuration having at least one sugar residue(s) bonded to two or more other sugar residues in the oligosaccharide moiety. Glycopyronosyl residues can optionally be described in relation to their ordered attachment to the 19C and 13C atoms of the steviol base, such as primary, secondary, tertiary residues, wherein a primary glycopyronosyl residue is directly bonded to the 19C and/or 13C atom, a secondary glycopyronosyl residue is directly bonded to a primary glycopyronosyl residue, a tertiary glycopyronosyl residue is directly bonded to a secondary glycopyronosyl residue, etc. Various known steviol glycosides are shown in FIG. 1, with reference to the steviol base structure of Formula I, and the R¹ and R² groups attached to the 19C and 13C positions of the steviol base, respectively. See, for example, Compendium of Food Additive Specifications (Joint FAO/WHO Expert Committee on Food Additives; 84th Meeting 2017, FAO JECFA Monographs).

Methods of the disclosure use a glycosyltransferase having UDP-xylose:19-steviol xylosyltransferase activity. A “glycosyltransferase” generally refers to an enzyme that is capable of transferring a saccharide moiety to an acceptor molecule, and a “steviol glycosyltransferase” or a “steviol glycoside glycosyltransferase” capable of transferring a saccharide moiety to either a steviol base acceptor or a steviol glycoside acceptor, respectively. The glycosyltransferase can also be described in terms of its activity of adding a particular saccharide moiety/moieties at a particular position/positions on the steviol or steviol glycoside molecule. In accordance with the methods, compositions, and engineered of the disclosure, a glycosyltransferase having UDP-xylose:19-steviol xylosyltransferase activity, such as a polypeptide of SEQ ID NO:1, is used. UDP-xylose:19-steviol xylosyltransferase activity refers to the enzymatic activity which transfers a xylose moiety from UDP-xylose to the 19C atom or a saccharide residue directly or indirectly attached to (and extending from) the 19C atom to the a steviol acceptor molecule. The glycosyltransferase may not exclusively have UDP-xylose:19-steviol xylosyltransferase activity and may (or may not) have a further and different glycosyltransferase activity, such as UDP-xylose:13-steviol xylosyltransferase activity, and/or UDP-glucose:19-steviol glucosyltransferase activity, and/or UDP-glucose:19-steviol glucosyltransferase activity, and/or UDP-rhamnose:19-steviol rhamnosyltransferase activity, and/or UDP- rhamnose:19-steviol rhamnosyltransferase activity. Glycosyltransferases of the disclosure can preferentially add xylose to the 19 position of the —steviol/steviol glycoside (over the 13 position). The ability of a glycosyltransferase to preferentially add xylose at the 19 position can be determined by performing an assay using a composition that includes glycosyltransferase and UDP-xylose, and analyzing the reaction product, wherein greater than 50% mol (or >60%, >70%, >80%, >90%, >95%, >97%, >98%, or >99%) of product is steviol glycoside xylosylated on the 19(c) position.

In aspects, a glycosyltransferase polypeptide having at least 50% identity to any of SEQ ID NOs:1-3 is used to transfer one or more xylose residues to the 19 carbon position of a steviol glycoside acceptor molecule. In the method, the steviol glycoside acceptor molecule has a single glucopyranose moiety or an oligosaccharide moiety including one or more glucopyranose residue(s) attached to the 19 carbon of the steviol glycoside. The steviol glycoside acceptor can be a compound of Formula I:

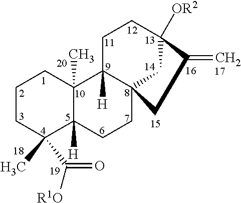

wherein R¹ comprises a glucose residue, and R² comprises one or more sugar residues or is a hydrogen. Exemplary steviol glycoside acceptors for xylosylation are stevioside, rebaudioside A (RebA), rebaudioside C (RebC), rebaudioside D (RebD), rebaudioside F, rebaudioside G, and dulcoside-A, with stevioside, RebA, and RebC being preferred substrates.

In some cases, the glycosyltransferase polypeptide of SEQ ID NO:1, 2, or 3 utilizes the activated xylose to attach a xylose to the glucopyranose residue at the 19 carbon position, resulting in an oligosaccharide including a glucopyranose residue glycosidically linked to a xylose residue. The enzymatic reaction can result in a compound of Formula II:

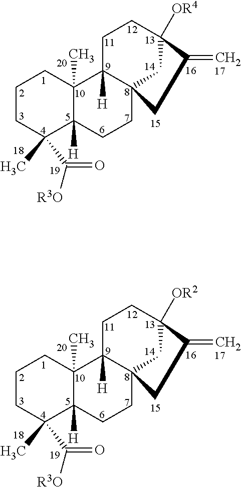

where R³ includes a xylose residue, and wherein R⁴ has the same chemistry as R² in the compound of Formula I, or R⁴ includes one or more additional sugar residues as compared to R². In some cases, the glycosyltransferase adds one or more xylose, glucose, or rhamnose moieties to the 13 carbon position (e.g., to a saccharide residue attached to the 13 carbon position). In aspects, R³ is an oligosaccharide moiety comprising a xylose residue, such as β-Glc-β-Xyl, which can be have a Glc2C→Xyl1C glycosidic linkage. For example, the xylosylated steviol glycoside can include attached to the 19 carbon of the steviol base an oligosaccharide moiety (R³) including β-Glc-β-Xyl, wherein β-Glc-β-Xyl can have a Glc2C→Xyl1C glycosidic linkage. An exemplary product of Formula II is SG-[13-β-Glc[(3→1)β-Glc](2→1)β-Glc(1-2)]-19-β-Glc(2→1)β-Xyl which is called rebaudioside DG (RebDG).

Glycosyltransferases constitute a family of enzymes that catalyze the transfer of a sugar (glycosyl) moiety to an acceptor molecule. Common acceptor molecules include saccharides and non-saccharides including polysaccharides, glycoproteins, glycolipids, and terpenes. Activated monomeric sugars, typically in the form of nucleoside diphosphate sugars, are used as substrates for the transfer of the sugar moiety to the acceptor molecule. A monosaccharide unit can be transferred to a hydroxyl or carboxyl moiety on a steviol or steviol glycoside molecule, or to a hydroxyl group on a glucose group that is attached to the steviol base. Glycosyltransferases can either invert the anomeric configuration of the sugar, such as forming a β-glucoside from UDP-glucose, or retain the anomeric configuration, such as forming an oc-glucoside from UDP-glucose.

Uridine diphosphate (UDP) glycosyltransferases (UGTs) are members of family 1 glycosyltransferases (GTs) that carry out glycosylation of natural products, in particular, the transfer of various sugars to plant natural products. There are also many UGT gene sub-families which are involved in the glycosylation of different types of plant products. E.g., see Wang, X. (2009) FEBS Letters 583:3303-3309. GTs have been classified into families and subfamilies based on sequence homology. See Li, et al., 2001 , J. Biol. Chem. 276:4338-4343. A superfamily of over 100 genes encoding UGTs, each containing a 42 amino acid consensus sequence, has been identified in the model plant Arabidopsis thaliana, and genes encoding UGTs have also been identified in several other higher plant species. Each GT family is composed of proteins related by sequence, as a consequence, by the type of protein folding. Accordingly, there is predictability within the family based on conservation of catalytic machinery. GT polypeptides have also been categorized into families by amino acid sequence similarities (e.g., see Coutinho et al., 2003). A list of GT families and members, which is actively curated, can be found at the Carbohydrate Active enZyme database at afmb.cnrs-mrs.fr/CAZY.

The crystal structures of some plant UGTs reveal the presence of a GT-B fold, one of two general folds of the GT superfamily of enzymes, and also that these UGTs have two N- and C-terminal domains with similar Rossmann-like folds. Some UGT crystal structures include those of M. truncatula UGT71G1; UGT85H2; UGT78G1; grape (Vitis vinifera) VvGT1 26; and A. thaliana UGT72B1. See, for example, Shao, H., et al. (2005) Plant Cell, 17:3141-3154); Modolo, L. V., et al. (2009), pp. J. Mol. Biol., 392:1292-1302; L. Li, L. et al. (2007) J. Mol. Biol., 370:951-963; Offen, W., et al. (2006) EMBO J., 25:1396-1405; Brazier, M. et al. (2007), Proc. Natl. Acad. Sci. 104:20238-20243; and Bourne, Y., and Henrissat, B. (2001) Curr. Opin. Struct. Biol., 11:593-600. See also Lin, M., et al. (Biochemical Engineering Journal, 59, 2020), which describes the modeled structure of SEQ ID NO:1 using a computational strategy that combines protein structure prediction, sequence optimization, and molecular dynamics simulation to improve the catalytic efficiency of the UDP-dependent glycosyltransferase SEQ ID NO:1 for the synthesis of rebaudioside D from rebaudioside A.

The enzyme of SEQ ID NO:1 belongs to the GT-B fold sub-family UGTs. SEQ ID NO:1 corresponds to a uridine 5′-diphospho (UDP) glycosyl transferase of 462 amino acids from Oryza sativa Japonica Group (see, for example, U.S. Pat. No. 9,631,215 (#152), and access number AC133334. Based on alignment with other EUGTs, amino acid residues of conserved domains including active sites, substrate binding pockets, and TDP binding sites can be identified in SEQ ID NO:1.

Methods of the disclosure use a polypeptide having at least 50% identity to SEQ ID NO:1. Preferably, the glycosyltransferase has at least 55%, 60%, 65%, 70% 75%, 80%, 85%, 90%, 91%, 92%, 93%, 94%, 95%, 96%, 97%, 98%, or 99% or greater identity to SEQ ID NO:1. In some aspects, the glycosyltransferase has 100% identity to SEQ ID NO:1.

An in silico modelling system was used to predict the interaction of polypeptide of SEQ ID NO:1 and ligands, which in turn was based on solved structures of homologs of SEQ ID NO:1. Data revealing amino acid residues having SEQ ID NO:1 that contact substrates was obtained for SEQ ID NO:1-RebA-UDP-xylose and SEQ ID NO:1-RebA-UDP-glucose by this in silico modelling. Data revealed the following amino acids involved in RebA-UDP-xylose (ligand) interaction: W22, L23, A24, F25, G26, H27, L28, L29, P30, L57, P59, D90, V91, P92, H93, D94, R95, P96, D97, M98, V99, D128, F130, L150, G151, 5152, M155, R190, M191, K192, R195, T196, K197, S199, 5200, G201, M202, 5203, L204, A205, R221, P249, P250, L251, Y277, A279, L280, G281, 5282, E283, V284, P285, A308, L309, R310, R338, W339, V340, P341, Q342, M343, L346, F354, H357, C358, G359, W360, N361, 5362, T363, E365, 1378, F379, G380, D381, Q382, and N385 (amino acid list “A”);. Data revealed the same amino acids were involved in RebA-UDP-glucose (ligand) interaction with the following exceptions: P59 and D128 are involved in UDP-xylose (ligand) interaction but not UDP-glucose interaction, and L149, S203, R310, and 1378 are involved in UDP-glucose (ligand) interaction but not UDP-xylose (ligand) interaction, although S203, R310, and I378 are still involved in RebA interaction.

Comparative sequence analysis revealed the following amino acids are highly conserved between SEQ ID NO:1 and other UGTs: M1, H16, V18, P21, W22, L23, A24, F25, G26, H27, P30, L34, L38, A39, G42, H43, S46, S49, T50, P51, N53, R56, L57, P58, V71, P76, L81, P82, A85, E86, T88, D90, A105, D107, L109, L117, D123, D128, W133, A138, A153, P180, E187, S200, R207, R221, S222, E225, E227, P241, G246, P249, W266, L267, Q270, S274, V275, Y277, V278, A279, G281, S282, E283, E293, L294, A295, G297, L298, E299, F305, W307, R310, L321, P322, G324, F325, R328, G333, V335, W339, P341, Q342, I345, L346, H348, V351, G352, F354, L355, T356, H357, G359, S362, E365, L373, L376, P377, D381, Q382, G383, N385, A386, R387, G395, V398, R400, D404, G405, F407, V412, A413, V419, and A433 (amino acid list “B”).

Comparative analysis also revealed the following amino acids that were highly conserved in UGTs but not in SEQ ID NO:1: M15, A35, Q36, S87, H102, R103, V143, F244, M248, A301, R304, A350, A415, S425, and Q430 (amino acid list “C”).

In some aspects the glycosyltransferase has less than 99.8% identity, but greater than 50%, 55%, 60%, 65%, 70% 75%, 80%, 85%, 90%, 91%, 92%, 93%, 94%, 95%, 96%, 97%, 98%, or 99% or greater identity to SEQ ID NO:1, and wherein the glycosyltransferase has one or more variant amino acid(s) that are not at a location in amino acid list “A,” not at a location in amino acid list “B,” or not at a location in both “A” and “B.” In some aspects, the glycosyltransferase based on SEQ ID NO:1 has one or more variant amino acid(s) that are at a location in amino acid list “C.”

In some aspects the glycosyltransferase having UDP-xylose:19-steviol xylosyltransferase activity includes one or more of the amino acid motifs: SEQ ID NO:1: WLAFGHLLP (SEQ ID NO:5), LPP; NDVPHDRPDMV (SEQ ID NO:6), DVF, LLGSAHM (SEQ ID NO:7); RMKLIRTKGSSGMSLA (SEQ ID NO:8); PPL; YVALGSEVP (SEQ ID NO:9); ALR; RWVPQMSIL (SEQ ID NO:10); FLTHCGWNSTIE (SEQ ID NO:11); IFGDQGPN (SEQ ID NO:12). These motifs correspond to the following amino acid positions in SEQ ID NO:1: 22-30, 57-59, 90-99, 128-130, 149-155, 190-205, 249-251, 277-285, 308-310, 338-346, 354-365, and 378-385.

Hughes and Hughes (DNA Sequence, 5:41-49, 1994) showed that the consensus motif for all glycosyltransferases could be refined and extended towards the N-terminus of the protein into a 44-amino acid consensus typical of secondary metabolism plant UGTs. This plant secondary product glycosyltransferase (PSPG) motif was later found in all cloned secondary metabolism UGTs from other plant species.

In some aspects the glycosyltransferase having UDP-xylose:19-steviol xylosyltransferase has one or more variant amino acid(s) at positions that are involved in RebA-UDP-glucose (ligand) interaction, but not RebA-UDP-xylose (ligand) interaction. As noted herein, data revealed the same amino acids were involved in RebA-UDP-glucose (ligand) interaction with the following exceptions P59 and D128 are involved in UDP-xylose (ligand) interaction but not UDP-glucose interaction, and L149, S203, R310, and I378 are involved in UDP-glucose (ligand) interaction but not UDP-xylose (ligand) interaction, although S203, R310, and I378 are still involved in RebA interaction. Therefore, relative to SEQ ID NO:1 or 2, the glycosyltransferase can have a variant amino acid at one or more of the following locations: L149, S203, R310, and I378W339.

The one or more amino acid variants can be a non-conservative substitution (more preferred), or a conservative substitution (less preferred). Such variation(s) can result in reduced interaction of the glycosyltransferase to interact with UDP-glucose, in turn favoring interaction with UDP-xylose and driving greater xylosylation of a steviol glycoside acceptor molecule. In other aspects, relative to SEQ ID NO:1 or 2, the glycosyltransferase can have a variant amino acid at one or more of the following locations: M15, A35, Q36, S87, H102, R103, V143, F244, M248, A301, R304, A350, A415, S425, and Q430 (amino acid list “C”).

In some aspects, the glycosyltransferase having UDP-xylose:19-steviol xylosyltransferase activity includes the following amino acid variation relative to SEQ ID NO:1: I152. For aspects using a polypeptide with less than 100% identity to any one of SEQ ID NOs:1-3, the difference in identity may be due to one or more amino acid substitutions in one or more region(s) of the polypeptide, for example, in regions outside of those understood to be important for active sites, substrate binding pockets, and TDP binding sites and/or outside of conserved regions based on alignment with one or more other UGTs of the same family as SEQ ID NO:1. Alternatively, if one or more amino acid substitutions, deletions, or additions are made to any of SEQ ID NOs:1-3, they are preferably made at locations that have a lower degree of identity between any of SEQ ID NOs:1-3 and other UGTs of the same family. In some aspects, the glycosyl transferase has one or more amino acid substitutions, deletions, or additions which cause the sequence to vary from the those of SEQ ID NO:1, SEQ ID NO:2, or SEQ ID NO:3 sequence while retaining certain sequence features. That is, if SEQ ID NO:1 is modified, it is modified at one or more amino acid locations outside of regions that are conserved and/or important for enzymatic functioning of the protein.

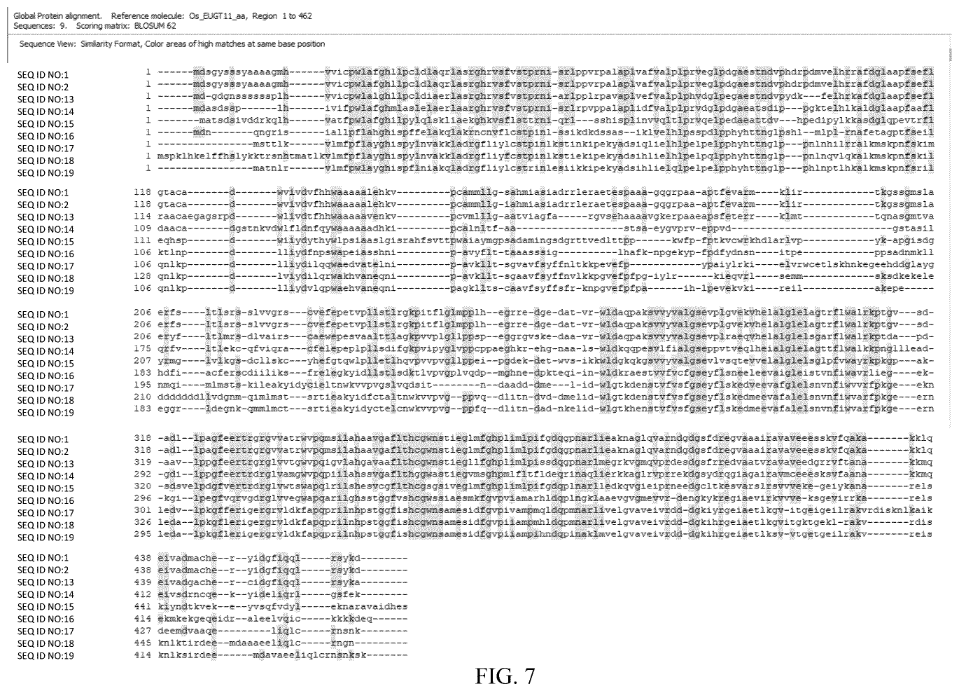

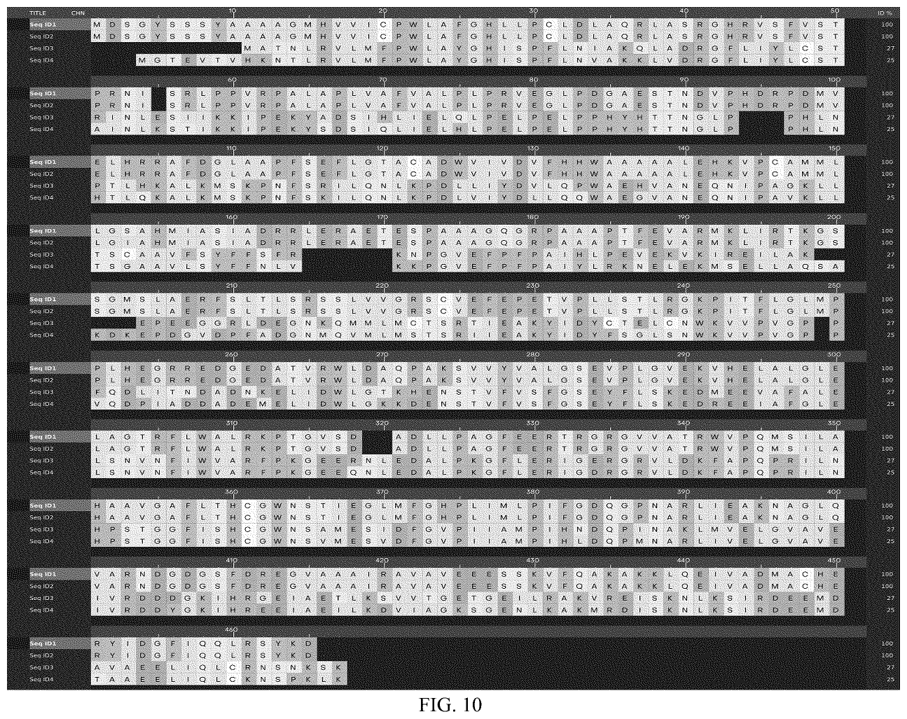

FIG. 7 shows an alignment of SEQ ID NO:1 with variants or other glycosyltransferases having homology to SEQ ID NO:1. As illustrated in the alignment, a column of shaded amino acids at a particular location represents amino acids in the SEQ ID NO:1 homologs that have identity or similarity to the corresponding amino acid in the SEQ ID NO:1 template. Likewise, FIG. 10 shows an alignment of SEQ ID NO:1 with SEQ ID NOs:2-4, with shading representing properties of different groups of amino acids.

In preferred aspects, any variant of SEQ ID NO:1, SEQ ID NO:2, or SEQ ID NO:3 either does not have a variant amino acid at that position or, if there is a variant, it is a conservative amino acid substitution. For example, using any alignment of two or more glycosyltransferases sequences, one of them being SEQ ID NO:1, SEQ ID NO:2, or SEQ ID NO:3, a variant glycosyltransferase may be generated by one or more amino acid substitutions made in a glycosyltransferase template such as SEQ ID NO:1, SEQ ID NO:2, or SEQ ID NO:3 at any amino acid position(s) aligning with positions 3-15, 18-20, etc., relative to SEQ ID NO:1, SEQ ID NO:2, or SEQ ID NO:3 (as reflected by non-shaded amino acids). Such substation may be preferably a conservative substitution, but non-conservative substitutions may be used if the functionality is not affected. Shaded amino acids in a template such as SEQ ID NO:1, SEQ ID NO:2, or SEQ ID NO:3, such as 1, 2, 16, 17, 21-24, etc., are preferably not subject to any substitution if a variant is used or prepared, or if they are at most a conservative amino acid substation is used.

“Conservative amino acid substitution(s)” or “conservative variation(s)” of a particular sequence refers to the replacement of one amino acid, or series of amino acids, with a functionally identical amino acid(s). Conservative substitution tables providing functionally similar amino acids are well known in the art, and include replacement of one amino acid with another having the same type of functional group or side chain polarity, size, shape or charge (e.g., aliphatic, aromatic, positively charged, negatively charged, polar, non-polar, positive polar, negative polar, uncharged polar, non-polar hydrophobic, ionizable acidic, ionizable basic, or sulfur containing residues). The following six groups each contain amino acids that can be conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); Histidine (H); 5) Isoleucine (I), Leucine (L), Methionine (M), Valine (V); and 6) Phenylalanine (F), Tyrosine (Y), Tryptophan (W).

Polypeptide sequence identity regions between SEQ ID NO:1, SEQ ID NO:2, or SEQ ID NO:3 and homologous polypeptides, can be understood using sequence alignment tools as described herein.

Glycosyl transferase polypeptides of the disclosure can also have deletions to one or more regions of the native glycosyl transferase polypeptide, wherein the deletions do not affect the polypeptides' glycosyl transferase activity. The deletions can be based on known information regarding the structure and function of native glycosyl transferases, including information regarding regions that are conserved and/or important for enzymatic functioning of the protein, for example such as the sequences (a)-(i) as described herein

The determination of “corresponding” amino acids from two or more glycosyl transferases can be understood by alignments of all or portions of their amino acid sequences. Sequence alignment and generation of sequence identity include global alignments and local alignments, which typically use computational approaches. In order to provide global alignment, global optimization forcing sequence alignment spanning the entire length of all query sequences is used. By comparison, in local alignment, shorter regions of similarity within long sequences are identified.

As used herein, an “equivalent position” means a position that is common to the two sequences (e.g., SEQ ID NO:1 and a different UGT sequence having the desired substitution(s)) that is based on a best alignment of the amino acid sequences of one glycosyl transferases or as alignment of the three-dimensional structures. Thus, either sequence alignment or structural alignment, or both, may be used to determine equivalence.

While amino acid positions described herein are with reference to the amino acid sequence of SEQ ID NO:1 or SEQ ID NO:2, corresponding positions in other glycosyl transferases for use in methods of the disclosure that do not have the same sequence length but that still can be aligned using the MUSCLE algorithm with an opening gap of 10 positions and an extending gap of 0.2 as implemented in the Schrodinger platform v 2020-4. As shown in FIG. 10, SEQ ID NO:1 or SEQ ID NO:2 have good alignment with SEQ ID NOs: 3 and 4, and therefore identification of corresponding positions in any of SEQ ID NOs: 3 and 4 relative to SEQ ID NO:1 and 3 can readily be understood. For example, in SEQ ID NO:3 the amino acid positions are shifted −10 from SEQ ID NOs:1 and 2 (i.e., position 11 in SEQ ID NO:1 is position 1 in SEQ ID NO:3) for the first 44 amino acids, and then are shifted −9 for the next 49 amino acids in SEQ ID NO:3.

In some modes of practice, the BLAST algorithm is used to compare and determine sequence similarity or identity. In addition, the presence or significance of gaps in the sequence which can be assigned a weight or score can be determined. These algorithms can also be used for determining nucleotide sequence similarity or identity. Parameters to determine relatedness are computed based on art known methods for calculating statistical similarity and the significance of the match determined. Gene products that are related are expected to have a high similarity, such as greater than 50% sequence identity. Exemplary parameters for determining relatedness of two or more sequences using the BLAST algorithm can be as follows.

In some modes of practice, an alignment is performed using BLAST (National Center for Biological Information (NCBI) Basic Local Alignment Search Tool) version 2.2.29 software with default parameters. A sequence having an identity score of XX% (for example, 80%) with regard to a reference sequence using the BLAST version 2.2.29 algorithm with default parameters is considered to be at least XX % identical or, equivalently, have XX % sequence identity to the reference sequence.

A global alignment can be used to align sequences with significant identity to SEQ ID NO:1, SEQ ID NO:2, or SEQ ID NO:3, in order to determine which corresponding amino acid position(s) in the target sequence (e.g., a glycosyltransferase ortholog) can be substituted with the one or more of the amino acids if a glycosyl transferase variant is used.

In aspects of the disclosure the method of forming the xylosylated steviol glycoside is carried out in a reaction composition. The reaction composition can be prepared to include desired types and amounts of the following reagents: (i) an steviol glycoside compound of Formula I (steviol glycoside acceptor); (ii) an activated xylose; and (iii) a glycosyltransferase having UDP-xylose:19-steviol xylosyltransferase activity, such as one having at least 50% identity to SEQ ID NO:l. The reaction composition can comprise components (i), (ii), and (iii), meaning other components can optionally be included in the reaction composition that are different than (i), (ii), and (iii). The reaction composition can include other components, such as non-activated sugar(s), cofactors, and a liquid or liquid mixture in which the reaction is carried out.

The reaction composition includes a steviol glycoside acceptor of Formula I:

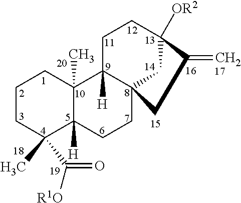

where R¹ includes a glucose residue, and R² includes one or more sugar residues or is hydrogen. In some cases, R¹ is a single glucose residue, such as β-Glc. Examples of steviol glycoside acceptors having single glucose residue at R¹ include steviol monoglucosyl ester (R¹=β-Glu, R²=—H), rubusodide (R¹=β-Glc, R²=β-Glc,), stevioside (R¹=β-Glc, R²=−β-Glc(1-2)β-Glc), rebaudioside A (RebA; R¹=β-Glc, R²=-β-Glc[β-Glc(1-3)]β-Glc(1-2)]), rebaudioside C (RebC; R¹=β-Glc, R²=−α-Rha(1-2)[β-Glc(1-3)]β-Glc), rebaudioside F (RebF; R¹=β-Glc, R²=β-Glc(1-2)[β-Xyl(1-3)]β- Glc), rebaudioside G (RebG; R¹=β-Glu, R²=−β-Glu(1-3)β-Glu), and dulcoside A (R¹=β-Glu, R²=α-Rha(1-2)β-Glu-). In some aspects, the steviol glycoside acceptor of Formula I is provided to the reaction composition in pure (e.g., greater than 99.9% (wt) of total steviol glycosides) or substantially pure form (e.g., greater than 99% (wt) of total steviol glycosides).

In some aspects, the steviol glycoside acceptor of Formula I can be provided to the reaction composition in mixture with one or more other steviol glycosides. For example, a mixture of steviol glycosides can be obtained from a process extracting steviol glycoside from a plant or plant cells. Alternatively, mixture of steviol can be obtained from engineered organisms capable of producing one or more steviol glycosides. For example, cell culture or fermentation medium can be used to obtain a mixture of steviol glycosides (see, for example, U.S. Pat. No. 9,631,215; WO2016/196321 (CAR0210/WO); WO2016/196345 (CAR0211/WO); WO2016/196368 (CAR0212/WO).

In aspects, in the reaction composition the steviol glycoside acceptor (compound of Formula I), which, for example, can be any one or a mixture of steviol monoglucosyl ester, stevioside, rebaudioside A, rebaudioside C, rebaudioside D, rebaudioside F, rebaudioside G, and dulcoside A. In addition to the steviol glycoside acceptor of Formula I, the reaction composition may include one or more other steviol glycoside(s) that are different than those of Formula I and that are not acceptor molecules for xylosylation. A reaction composition including a mixture of acceptor and non-acceptor steviol glycosides can be obtained from leaf (e.g., steviol leaf) extract.

In other aspects, the reaction composition can be enriched for one or more steviol glycoside acceptor(s) of Formula I. For example, in the reaction composition the steviol glycoside acceptor(s) of Formula I constitutes more than 50% (mol), 75% (mol) or greater, 85% (mol) or greater, 90% (mol) or greater, 92.5% (mol) or greater, 95% (mol) or greater, 97% (mol) or greater, 98% (mol) or greater, 99% (mol) or greater, 99.5% (mol) or greater, or 99.9% (mol) or greater, or essentially all of steviol glycoside therein. In some aspects, the steviol glycoside component of the reaction mixture consists essentially of one or more compounds of Formula I, meaning that other steviol glycosides not of Formula I can be present in the steviol glycoside component of the reaction mixture but in very small quantities (less than 1% (wt) of total steviol glycosides). In yet other aspects, the steviol glycoside component of the reaction mixture consists of one or more compounds of Formula I, meaning that other steviol glycosides not of Formula I are not in any detectable amount in the steviol glycoside component of the reaction mixture.

In some aspects, the reaction composition comprises rebaudioside A which constitutes more than 50% (mol), 75% (mol) or greater, 85% (mol) or greater, 90% (mol) or greater, 92.5% (mol) or greater, 95% (mol) or greater, 97% (mol) or greater, 98% (mol) or greater, 99% (mol) or greater, 99.5% (mol) or greater, or 99.9% (mol) or greater, or essentially all of steviol glycoside therein. In other aspects, the steviol glycoside component of the reaction composition consists of rebaudioside A.

In aspects, the steviol glycoside (SG) acceptor, which can be any one or a mixture of steviol glycoside acceptor, is present in the reaction composition in an amount where the SG acceptor is soluble in the reaction mixture. For example, the (SG) acceptor can be present in the range of about 0.01 to about 1.0 molar (e.g., about 0.01 μmoles to about 1 μmoles of SG acceptor per 100 μL reaction volume), about 0.025 to about 0.5 molar, or about 0.05 to about 0.2 molar.

The composition also includes a glycosyltransferase polypeptide having UDP-xylose:19-steviol xylosyltransferase activity. In aspects the glycosyltransferase comprises the following amino acids of amino acid list “A” and/or amino acid list “B” relative to SEQ ID NO:1 or 2. The glycosyltransferase of the disclosure can be a polypeptide having at least 50% identity to SEQ ID NO:1 or 2, and preferably at least 60%, 75%, 85%, 90%, 95%, 98%, or 99% or greater identity to SEQ ID NO:1 or SEQ ID NO:2. U.S. Pat. No. 9,631,215 describes the purification of a 6HIS- or GST-tagged SEQ ID NO:1 from a recombinant E. coli strain expressing this fusion protein.

In aspects, the glycosyl transferase polypeptide, is present in the reaction composition in an amount in the range of about 0.25% (wt) to about 10% (wt) (e.g., about 0.25 μg to about 10 μg protein 100 μL reaction volume), about 0.5% (wt) to about 5% (wt), or about 1% (wt) to about 3% (wt).

Optionally, the amounts of steviol glycoside (SG) acceptor and glycosyl transferase polypeptide present in the reaction composition can be described with reference to one another. For example, the reaction composition can have a glycosyl transferase polypeptide (wt) to steviol glycoside (SG) acceptor (mol) ratio in the range of 25 μg: 1 μmol to 5 μg: 1 μmol, in the range of 15 μg: 1 μmol to 7.5 μg: 1 μmol, or about 10 μg: 1 μmol.

The reaction composition also includes an activated xylose which is preferably in the form of UDP-xylose (uridine[5′]diphospho-α-D-xylopyranoside), which is commercially available (e.g., Biosynth Carbosynth, UK; Sigma-Aldrich). In aspects, UDP-xylose is present in the reaction composition in an amount in the range of about 0.02 to about 2.0 molar (e.g., about 0.02 μmoles to about 2 μmoles of UDP-xylose per 100 μL reaction volume), about 0.05 to about 1.0 molar, or about 0.1 to about 0.4 molar. UDP-xylose has the following chemical structure:

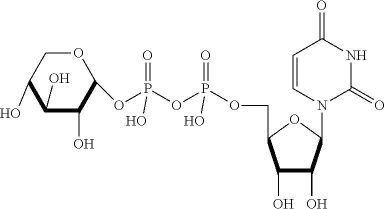

Optionally, the amounts of UDP-xylose and steviol glycoside (SG) acceptor, or the amounts of UDP-xylose and glycosyl transferase polypeptide, or present in the reaction composition can be described with reference to one another. For example, in some aspects, the reaction composition includes a molar excess of UDP-xylose (UDP-X) to the steviol glycoside compound of formula I (SGI), or a UDP-X:SGI molar ratio in the range of 1.1:1 to 100:1, 1.1:1 to 10:1, 1.2:1 to 5:1, or 1.5:1 to 3:1. In some aspects, the reaction composition includes a glycosyl transferase polypeptide (wt) to UDP-X (mol) ratio in the range of 50 μg: 1 μmol to 10 μg: 1 μmol, in the range of 25 μg: 1 μmol to 15 μg: 1 μmol, or about 20 μg: 1 μmol;

The reaction composition can also include one or more non-activated sugar components to promote the reaction. For example, the composition can include a non-activated sugar, such as sucrose, to facilitate stabilization of the enzymatic reaction. Other non-activated sugars include maltose, trehalose, glucose, and starch-hydrolysates such as glucose syrup and maltodextrin. A non-activated sugar can be used in an amount in the range of about 10 mM to about 0.5 M, or about 50 mM to about 0.2 M. Optionally, the amounts of non-activated sugar and steviol glycoside (SG) acceptor can be described with reference to one another. For example, in some aspects, the reaction composition includes a molar excess of non-activated sugar to the steviol glycoside compound of formula I (SGI) in the range of 0.1:1 to 1:1, 1:1 to 5:1, 2:1 to 5:1; 5:1 to 10:1, or 10:1 to 20:1.

The composition can also include a salt that provides a divalent cation which can be used as a cofactor for the glycosyl transferase polypeptide. Exemplary divalent metal salts include magnesium and/or manganese salt(s), which can be used in an amount in the range of about 0.5 mM to about 5 mM or about 1 mM to about 4 mM in the reaction mixture.

The reaction can be carried out in at a neutral or slightly basic pH, such as a pH in the range of 4.0-8.0, 6.8-7.8, or 7.1-7.5, using a buffer such as one including a citrate salt, a phosphate salt, or tris(hydroxymethyl)aminomethane (Tris).

In some aspects the reaction composition consists essentially of (i) the steviol glycoside compound of Formula I (steviol glycoside acceptor), (ii) the activated xylose; (iii) the polypeptide having at least 50% identity to SEQ ID NO:1, (iv) a non-activated sugar, (v) a salt of a divalent cation, and (vii) a buffer.

Reaction can be carried out at a desired temperature and time to promote xylosylation of the steviol glycoside acceptor molecule. For example, the reaction is carried out for at least one hour, and up to about 10 days, such as an amount of time in the range of 1 hour to 24 hours, in the range of 1 hour to 12 hours, in the range of about 12 hours to about 7 days, or about 1 day to about 5 days. The reaction can be carried out at a temperature in the range of about: 5-95° C., 25-80° C., 25-40° C., 30-40° C., 40-50° C., 50-60° C., 60-70° C., 70-80° C., or 25-35° C., or 28-32° C.

As a result of the reaction, a “product composition” is formed, which includes one or more xylosylated steviol glycosides remaining reaction components including the polypeptide and any excipient component(s) or component(s) resulting from the reaction. In some cases, the product composition can include some amount of unreacted steviol glycoside acceptor reactant.

Following reaction, the product composition can be subjected to a refinement to separate components of the reaction mixture. Xylosylated steviol glycoside products can be purified by methods such as crystallization as described herein, or by using a reverse phase chromatography column. Hydrophilic components of the reaction mixture can be removed with water and the xylosylated steviol glycoside compounds can be removed by elution with a solvent like methanol. Further, xylosylated steviol glycoside compounds can be further resolved using preparative HPLC (e.g., see WO 2009/140394).

The reaction composition can provide a xylosylated steviol glycoside compound of Formula II:

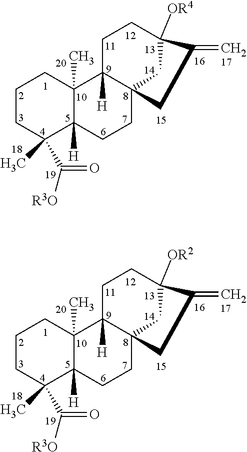

where R³ includes a xylose residue, and wherein R⁴ includes one or more sugar residues or is hydrogen. Exemplary compounds include those wherein R³ is an oligosaccharide moiety comprising a xylose residue, such as β-Glc-β-Xyl and having a Glc2C→Xyl1C glycosidic linkage. In aspects, the xylosylated steviol glycoside is selected from one or more of the following compounds:

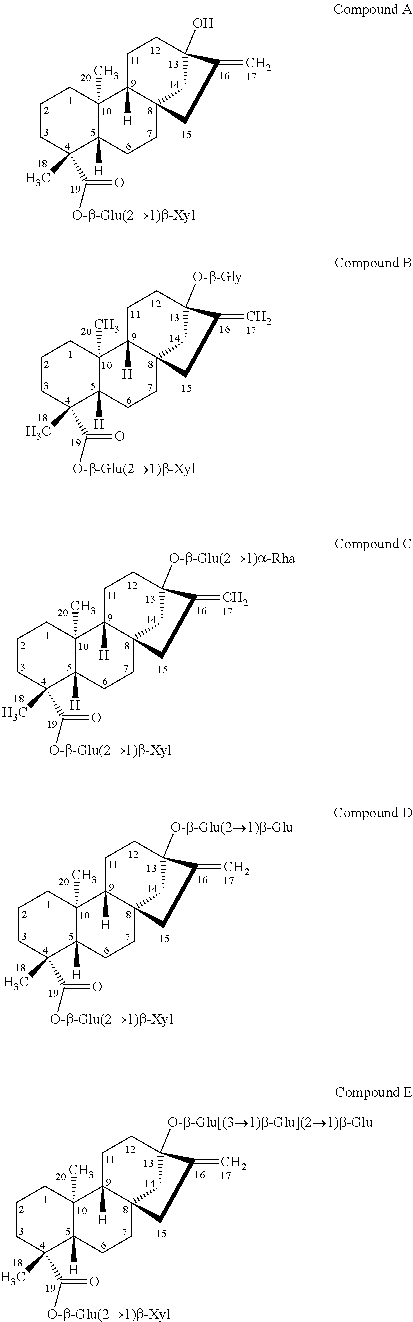

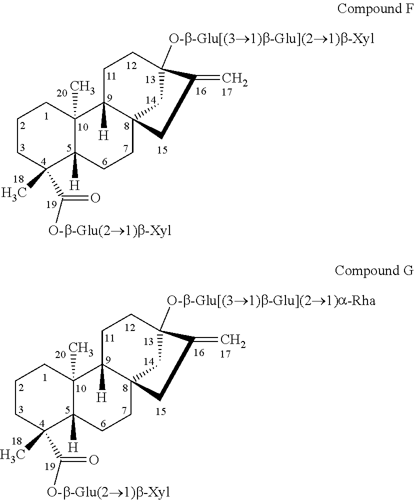

The chemical structure of Compound E (RebDG) is shown below:

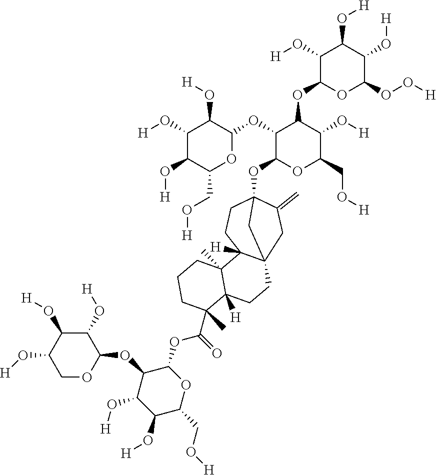

The reaction can also provide high yield of the xylosylated steviol glycoside. In particular, in the reaction composition, more than 50%, more than 60%, more than 65%, more than 75%, or more than 80% of the steviol glycoside compound of Formula I is converted to xylosylated steviol glycoside compound of Formula II.

The reaction can also provide a product composition that includes one or more compounds of Formula II as described herein. The type and relative amount(s) of xylosylated steviol glycoside product(s) can depend on the type and relative amount(s) of steviol glycoside acceptor(s) used in the reaction composition.

In some aspects the product composition includes a mixture of steviol glycoside components that includes one, two, or three or more xylosylated steviol glycosides of Compounds A-F. In some aspects the product composition includes a mixture of steviol glycoside components that includes at least the xylosylated steviol glycosides of Compounds E and/or F. In some aspects, Compounds E and/or F are the predominant steviol glycoside component in the product composition, meaning that the compound is present in an amount greater than any other steviol glycoside in the product composition. In other aspects, two or more Compounds E and F are present the predominant steviol glycoside component in the product composition, meaning that the compound is present in an amount greater than any other steviol glycoside in the product composition In some aspects Compounds E and/or F are in the product composition in an amount of greater than 50%, greater than 60%, greater than 65%, greater than 75%, greater than 80%, greater than 85%, greater than 90%, or greater than 95% of the total amount of steviol glycoside in the composition.

Following reaction, the product composition can be subjected to a refinement to separate components of the reaction mixture. The glycosylated steviol glycoside products can be purified using methods such as crystallization or by using a reverse phase chromatography column. Hydrophilic components of the reaction mixture can be removed with water and the glycosylated steviol glycoside compounds can be removed by elution with a solvent like methanol. Further, glycosylated steviol glycoside compounds can be further resolved using preparative HPLC (e.g., see WO 2009/140394).

In some aspects an engineered cell is used to prepare the xylosylated steviol glycoside compound of Formula II. The engineered cell can have a pathway that is capable of making a steviol glycoside acceptor compound of Formula I, and can express a glycosyltransferase polypeptide having UDP-xylose:19-steviol xylosyltransferase activity, such as a polypeptide having at least 50% identity to SEQ ID NO:1. The cell also may express a pathway for forming activated xylose, such as UDP-xylose.

In aspects, the engineered cell includes a pathway to steviol, which is a precursor for compounds of Formula I, which in turn can be used as substrates to form compounds of Formula II using the glycosyltransferase polypeptide having UDP-xylose:19-steviol xylosyltransferase activity, such as a polypeptide having at least 50% identity to SEQ ID NO:1 and activated xylose. The cell can be engineered to provides the steviol glycoside compound of Formula I in an amount that is greater than an amount of steviol glycoside not of Formula I that may be formed in the cell. The cell also expresses a glycosyltransferase polypeptide having UDP-xylose:19-steviol xylosyltransferase activity, such as a polypeptide having at least 50% identity to SEQ ID NO:1, wherein the polypeptide is capable of transferring xylose from the activated xylose to the compound of Formula Ito form a xylosylated steviol glycoside compound of Formula II. Exemplary engineered cells include those that are engineered yeast, bacteria, and fungus.

The terpenoid compounds isopentenyl diphosphate (IPP) and dimethylallyl diphosphate (DMAPP) can serve as chemical precursors to steviol glycosides in an engineered cell. Some organisms, including plants, insect, and some microbial species, have a mevalonate (MVA) pathway that converts acetyl-CoA through a series of chemical intermediates to IPP and DMAPP. Some organisms produce IPP and DMAPP through the non-mevalonate pathway (also known as the methyl D-erythritol 4-phosphate or MEP pathway) starting with glyceraldehyde-3-phosphate (G3P) and pyruvate (PYR).

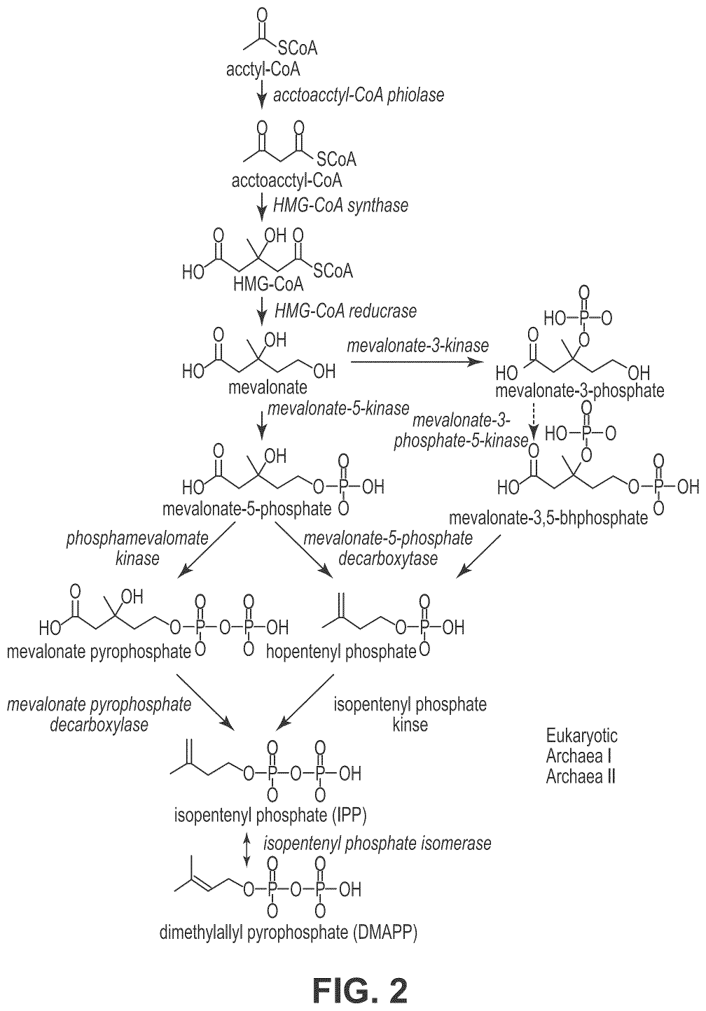

FIG. 2 shows a representative mevalonate pathway. In this pathway the mevalonate pathway genes include: (a1) acetoacetyl CoA thiolase (EC 2.3.1.9), (b1) 3-hydroxy-3-methylglutaryl-coenzyme A (HMG-CoA) synthase (EC 4.1.3.5); (c1) HMG-CoA reductase (EC 1.1.1.34); (d1) mevalonate kinase (EC 2.7.1.36); (e1) phosphomevalonate kinase (EC 2.7.4.2); and (f1) mevalonate diphosphate decarboxylase (EC 4.1.1.33). Enzymes of the mevalonate pathway converts acetyl-CoA to IPP as follows: acetyl-CoA→acetoacetyl-CoA→3-hydroxy-3-methylglutaryl-CoA→mevalonate→mevalonate-5-phosphate→mevalonate-5-pyrophosphate→IPP. Alternatively, the pathway can include enzymes from Archaea using mevalonate-3-kinase which is converts mevalonate to mevalonate-3-phosphate, then mevalonate-3-phosphate-5-kinase converting mevalonate-3-phosphate to mevalonate-3,5-biphosphate, and mevalonate-5-phosphate decarboxylase converting mevalonate-3,5-biphosphate to isopentyl phosphate, and then isopentyl phosphate kinase converting isopentyl phosphate to IPP.

Some host cells may not include any of the necessary enzymes for a mevalonate pathway, whereas some cells may include some, but not all, mevalonate pathway genes, whereas some host cells may naturally include all of the genes for the mevalonate pathway. In some cases, host cells that do not include any, or include some, can be engineered to include those missing mevalonate pathway genes. The yeast Saccharomyces cerevisiae naturally expresses genes of the mevalonate pathway, but can be engineered to provide increased expression of those pathway genes.

In some aspects, a prokaryotic cell is engineered with the mevalonate pathway. Martin, V. J., et al. describes Engineering a mevalonate pathway in Escherichia coli for production of terpenoids (Nature Biotechnology 21:796-802, 2003). Wang, J., et al. describes Engineering of a highly efficient Escherichia coli strain for mevalonate fermentation through chromosomal integration (Appl. Environ. Microbiol. 82:7176 —7184, 2016).

In some aspects, a eukaryotic cell is engineered with the mevalonate pathway, or engineered to provide greater amounts of IPP through modification of mevalonate pathway genes. For example, Gold, N. D., et al. describes a combinatorial approach to study cytochrome P450 enzymes for de novo production of steviol glucosides in baker's yeast. ACS Synth. Biol. 7: 2918-2929 (2018).

Alternatively, a non-mevalonate (MEP) pathway can be used to provide IPP and DMAPP as precursors to steviol production. Theoretically, the MEP pathway is more energetically efficient generally because it loses less carbon as CO₂ as compared to the MVA pathway (MEP pathway: 1 CO₂/IPP; MVA pathway: 4 CO₂/IPP; sugar as carbon source).

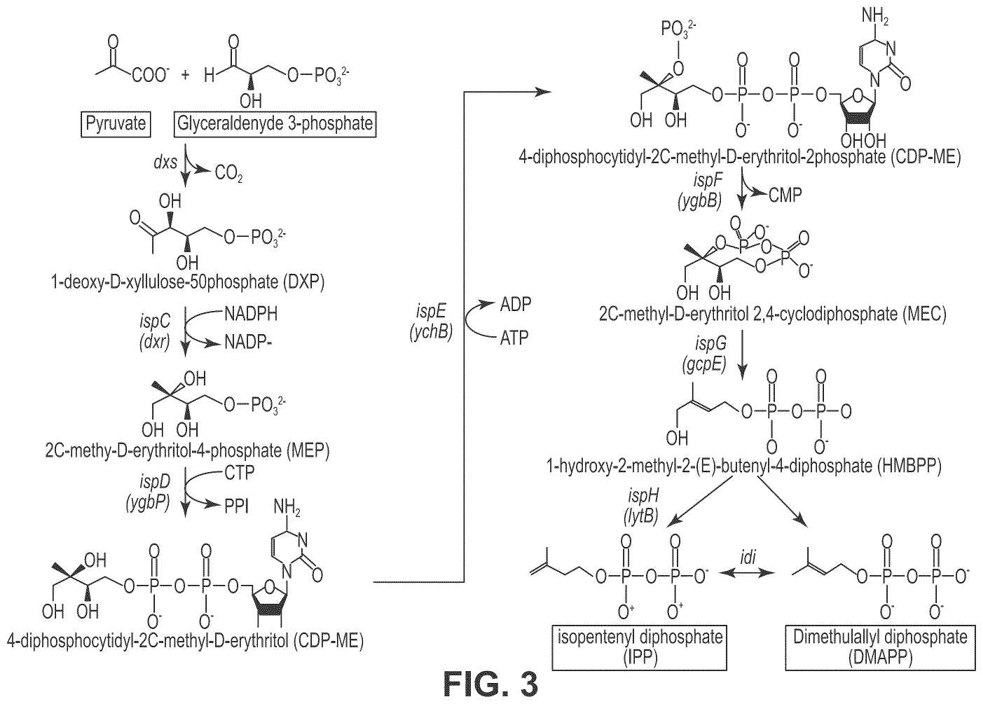

In particular, in the non-mevalonate (MEP) pathway compounds isopentenyl diphosphate (IPP), dimethylallyl diphosphate (DMAPP) are generated through a series of intermediates leading from glyceraldehydes-3-phosphate (G3P) and pyruvate (PYR), and a number of enzymes are responsible for this conversion. FIG. 3 shows a representative non-mevalonate pathway. Enzymes involved in a biosynthetic pathway from G3P and PYR to IPP and DMAPP include (a2) 1-deoxy-D-xylulose-5-phosphate synthase (DXS), (b2)1-Deoxy-D-xylulose-5-phosphate reductoisomerase (ispC)-, (c2) 4-diphosphocytidyl-2C-methyl- D-erythritol synthase (IspD), (d2) 4-diphosphocytidyl-2-C-methyl-D-erythritol kinase (IspE), (e2) 2C-Methyl-D-erythritol-2,4-cyclodiphosphate Synthase (IspF), (f2)1-hydroxy-2-methyl-2-(E)-butenyl-4-diphosphate synthase (IspG), (g2) 4-hydroxy-3-methyl-2-(E)-butenyl-4-diphosphate reductase (IspH), and (h2) isopentenyl-diphosphate isomerase (IDI).

U.S. Pat. No. 9,284,570 describes a method for producing steviol or steviol glycoside in E. coli, that uses an upstream methylerythritol pathway (MEP) that produces isopentyl pyrophosphate (IPP) and dimethylallyl pyrophosphate (DMAPP).

The yeast Saccharomyces cerevisiae does not naturally express genes of the MEP pathway, but can optionally be engineered to provide MEP pathway genes.

In some aspects, the engineered cell can include one or more modifications to increase the flux from acetyl-CoA to IPP and/or DMAPP, thereby providing an increased pool of IPP and/or DMAPP for use in a pathway to steviol. The modifications can include, for example, increasing expression or activity of one or more mevalonate pathway enzymes (a1)-(f1), such as by placing a nucleic acid encoding an enzyme that is homologous or heterologous to the cell under the control of a promoter that provides increased expression, using multiple copies of the nucleic acid, and/or using a heterologous enzyme, a variant enzyme (e.g., one including one or more amino acid substitutions), or a variant heterologous enzyme that provides a higher level of enzymatic activity as compared to the native enzyme.

The methods of the disclosure for producing xylosylated steviol glycoside(s) that use engineered cells can include cells that have one or more genetic modifications that increase the flux from G3P and PYR to IPP and/or DMAPP, thereby providing an increased pool of IPP and/or DMAPP for use in a pathway to steviol. The modifications can include, for example, increasing expression or activity of one or more enzymes (a2)-(h2), such as by placing a nucleic acid encoding an enzyme that is heterologous to the host cell under the control of a promoter that provides increased expression, using multiple copies of the nucleic acid, and/or using a heterologous enzyme, a variant enzyme (e.g., one including one or more amino acid substitutions), or a variant heterologous enzyme that provides a high levels of enzymatic activity.

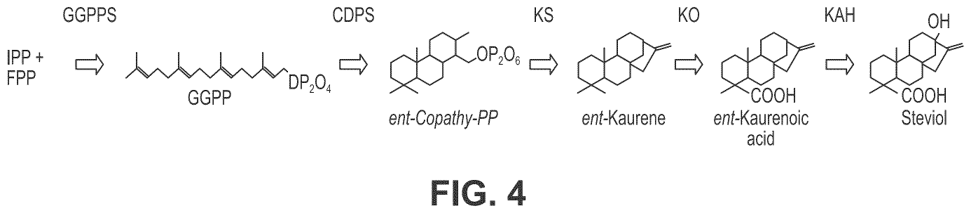

The methods of the disclosure for producing xylosylated steviol glycoside(s) can use engineered cells can also include a pathway to convert IPP and/or DMAPP and farnesyl pyrophosphate (FPP) to steviol. For example, and with reference to FIG. 4, in some aspects the engineered cells can include exogenous nucleic acids expressing the following enzymes: (a3) geranyl geranyldiphosphate synthase (GGPPS), (b3) copalyl diphosphate synthase (CPS), (c3) kaurene synthase (KS), (d3) kaurene oxidase (KO), and (e3) kaurenoic acid 13-hydroxylase (KAH). Enzymes of the mevalonate pathway convert IPP and/or DMAPP to steviol as follows: IPP/DMAPP→geranyl geranyldiphosphate→copalyl diphosphate→kaurene→kaurenoic acid→steviol. Exogenous nucleic acids encoding enzymes (a3)-(e3) that are heterologous to the yeast cell can be placed under the control of a promoter that provides increased expression, using multiple copies of the nucleic acid, and/or using a variant enzyme (e.g., one including one or more amino acid substitutions), or a variant heterologous enzyme that provides a high levels of enzymatic activity.

U.S. Pat. No. 9,284,570 describes a method for producing steviol or steviol glycoside in E. coli, that uses an upstream methylerythritol pathway (MEP) that produces isopentyl pyrophosphate (IPP) and dimethylallyl pyrophosphate (DMAPP), and a downstream pathway that produces steviol or steviol glycoside from said IPP and DMAPP, which expresses copalyl diphosphate synthase (CPS), kaurene synthase (KS), a geranylgeranyl diphosphate synthase (GGPPS) kaurenoic acid 13-hydroxylase (KAH) and kaurene oxidase (KO), and optionally one or more Stevia UDP glycosyl transferase enzymes.

The engineered cell also expresses the glycosyl transferase polypeptide having UDP-xylose:19-steviol xylosyltransferase activity, such as a polypeptide having at least 50% identity to SEQ ID NO:1, and preferably at least 60%, 75%, 85%, 90%, 95%, 98%, or 99% or greater identity to SEQ ID NO:1.

Regulation of the expression of heterologous genes can be controlled using desired promoters, terminators, and gene copy number.

In some aspects, the engineered cell is engineered to make more Reb A, Reb C, or stevioside than other steviol glycosides.

The engineered cell can include one or more uridine diphosphate (UDP) glycosyltransferases (UGTs) that are different than SEQ ID NO:1, or a homolog or variant of SEQ ID NO:1, and that mediate the transfer of glycosyl residues from activated nucleotide sugars to steviol acceptor molecules.

Exemplary UDP-glucosyltransferases other than one having SEQ ID NO:1 can be any UDP-glucosyltransferase capable of adding at least one glucose unit to the steviol and or steviol glycoside substrate to provide the target steviol glycoside. In one aspect, the engineered cell can include one or more UDP-glucosyltransferase selected from group UGT74G1, UGT85C2, UGT76G1, UGT91d2, and also UGTs having substantial (>85%) identity to these polypeptides. An engineered cell can include one or more exogenous nucleic acid molecule(s) that code for these UGTs.

The engineered cell can also include one or more UDP-glucose recycling enzyme(s) under heterologous gene control and/or one or more UGT. An exemplary UDP-glucosyltransferase capable of adding at least one glucose unit to rubusoside to form stevioside is UGT91d2. An exemplary UDP-glucosyltransferase capable of adding at least one glucose unit to stevioside to form rebaudioside A is UGT76G1. An exemplary UDP-glucosyltransferase capable of adding at least one glucose unit to rebaudioside A to form rebaudioside D is UGT91d2. An exemplary UDP-glucosyltransferase capable of adding at least one glucose unit to rebaudioside D to form rebaudioside M is UGT76G1.

Exemplary publications that describe engineered microorganisms for steviol glycoside production and steviol glycoside pathway enzymes include, for example, US2014/0357588, WO2014/193934, WO2014/193888, and WO2014/222227.

In one aspect, an engineered cell useful for the production of steviol glycosides expresses some or all of the following enzymes: geranylgeranyl diphosphate synthase (GGPPS), ent-copalyl diphosphate synthase (CDPS), kaurene oxidase (KO), kaurene synthase (KS); steviol synthase (KAH), cytochrome P450 reductase (CPR), UGT74G1, UGT76G1, UGT91d2, and a polypeptide of SEQ ID NO:1. WO 2014/122227 describes an engineered yeast strain that express these enzymes. The UGT74G1 enzyme functions as a uridine 5′-diphospho glucosyl:steviol 19-COOH transferase and a uridine 5′-diphospho glucosyl: steviol-13-O-glucoside 19-COOH transferase. The UGT76G1 enzyme is a stevia uridine diphosphate dependent glycosyltransferase that catalyzes several glycosylation reactions on the steviol backbone. The UGT76G1 enzyme can catalyze glycosylation of steviol and steviol glycosides at the 19-0 position or the 13-0 position.

The UGT91d2 enzyme can function as a uridine 5′-diphospho glucosyl: steviol-13-O-glucoside transferases (also referred to as a steviol-13-monoglucoside 1 ,2-glucosylase), transferring a glucose moiety to the C-2′ of the 13-O-glucose of the acceptor molecule, steviol-13-O-glucoside, or as uridine 5′-diphospho glucosyl: rubusoside transferases transferring a glucose moiety to the C-2′ of the 13-O-glucose of the acceptor molecule, rubusoside, to produce stevioside. The enzyme of SEQ ID NO:1 also can transfer a glucose moiety to the C-2′ of the 19-O-glucose of the acceptor molecule, rubusoside, to produce a 19-O-1,2-diglycosylated rubusoside.

Activated xylose is provided in the engineered cell, such as by feeding activated xylose to the cell from an external source, or the engineered cell is capable of making activated xylose. The polypeptide transfers xylose from the activated xylose to the compound of Formula I to form a xylosylated steviol glycoside compound of Formula II.

An “engineered cell” refers to a host cell having at least one exogenous DNA sequence that is introduced into the cell, either integrated into the cell's genome or present on an extrachromosomal construct, such as a plasmid or episome. The term “exogenous” refers to a molecule, such as a nucleic acid, or an activity, such as an enzyme activity, that is introduced into the host cell. An exogenous nucleic acid can be introduced into the host cell by well-known techniques and can be maintained external to the hosts chromosomal material (e.g., maintained on a non-integrating vector), or can be integrated into the cell's chromosome, such as by a recombination event. Generally, the genome of an engineered cell is augmented through the stable introduction of one or more recombinant genes. An exogenous nucleic acid can encode an enzyme, or portion thereof, that is either homologous or heterologous to the cell. An exogenous nucleic acid can be in the form of a “recombinant gene or DNA construct” referring to a nucleic acid that is in one or more ways manipulated through molecular techniques to be in a form that does not naturally exist.

The term “heterologous” (e.g., “non-native”) refers to a molecule or activity that is from a source that is different than the referenced molecule or organism. Accordingly, a gene or protein that is heterologous to a referenced organism is a gene or protein not found in that organism. In the context of the disclosure, a “heterologous glycosyltransferase” refers to a glycosyltransferase polypeptide that is different from any glycosyltransferase polypeptide that may be native to the host organism. For example, a specific glycosyltransferase gene found in a first species and exogenously introduced into a host cell organism that is different than the first species is “heterologous” to the host cell.

In some aspects, the engineered cell that produces xylosylated steviol glycoside(s) is a prokaryotic cell. Exemplary bacteria that can be used for hosts for exogenous DNA constructs encoding steviol glycoside pathway enzymes, include, but are not limited to species of Escherichia, Streptococcus, Lactobacillus, Pseudomonas, Lactococcus , Streptomyces, Bacillus, Clostridium, Ralstonia, Mycobacterium, Agrobacterium, Geobacter, , Zymonas, Acetobacter, Citrobacter, Synechocystis, Rhizobium, Corynebacterium, Xanthomonas, Alcaligenes, Aeromonas, Azotobacter, Comamonas , Rhodococcus, Gluconobacter, , Acidithiobacillus , Microlunatus , Geobacillus , Arthrobacter, Flavobacterium, Serratia, Saccharopolyspora, Therms, Stenotrophomonas, Chromobacterium, Sinorhizobium, Saccharopolyspora, and Pantoea. An exemplary bacterial species is Escherichia coli.

In some aspects, the engineered cell that produces xylosylated steviol glycoside(s) is a eukaryotic cell.

For example, various yeast host cells engineered to provide a pathway to one or more xylosylated steviol glycosides. Such cells can be transformed with one or more DNA construct(s) encoding enzymes for xylosylated steviol glycoside synthesis. Exemplary yeast that can be used for hosts for exogenous DNA constructs encoding steviol glycoside pathway enzymes, include, but are not limited to species of Agaricus, Aspergillus, Brettanomyces Candida, Fusariumm, Gibberella, Eluyveromyces, Hansenula, Humicola, Issatchenkia, Kloeckera (Hanseniaspora), Kluyveromyces, Laetiporus, Lentinus, Lipomyces, Pachysolen, Phaffia, Phanerochaete, Physcomitrella, Pichia (Hansenula), Rhodotorula, Saccharomycete, Saccharomyces, Schizosaccharomyces, Sphaceloma, Torulopsis, Torulaspora, Trichosporon Xanthophyllomyces Yamadazyma, Yarrowia, and Zygosaccharomyces. Exemplary species are Arxula adeninivorans, Ashbya gossypii, Candida albicans, Candida boidinii, Candida glabrata, Candida krusei, Cyberlindnera jadinii, Eluyveromyces lactis, Hansenula polymorpha, Issatchenkia orientalis, Pichia pastoris, Saccharomyces cerevisiae, Schizosaccharomyces pombe, Xanthophyllomyces dendrorhous, and Yarrowia hpolytica. Further, host cells can also include genetic modifications other than those of the steviol glycoside pathway that may provide improved performance during fermentation.

The engineered yeast can use an auxotrophic marker suitable for selecting for a transformant having a nucleic acid encoding a steviol glycoside pathway enzyme. The host yeast can include modifications (deletions, etc.) in one or more genes that control auxotrophies, such as LYS2, LEU2, HIS3, URA3, URA5, and TRP1. Using a host cell having a desired genetic background for introduction of one or more exogenous genes, one or more gene construct(s) is introduced into a cell to integrate into the genome, or to be stably maintained and allow for expression. Methods for introducing a gene construct into a host cell include transformation, transduction, transfection, co-transfection, and electroporation. In particular, yeast transformation can be carried out using the lithium acetate method, the protoplast method, and the like. The gene construct to be introduced may be incorporated into a chromosome in the form of a plasmid, or by insertion into the gene of a host, or through homologous recombination with the gene of a host. The transformed yeast into which the gene construct has been introduced can be selected with a selectable marker (for example, an auxotrophic marker as mentioned above). Further confirmation can be made by measuring the activity of the expressed protein, or the production of a bioproduct such as a steviol glycoside.

The transformation of exogenous nucleic acid sequences including the steviol pathway genes can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of the introduced nucleic acid sequences or their corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

The methods of the disclosure for producing steviol glycoside(s) by cell culture can use engineered cells having a pathway to convert steviol to a xylosylated steviol glycoside of Formula II. If more than one steviol glycoside pathway enzymes is present in the engineered yeast, the yeast may be able to produce different steviol glycosides, wherein at least one of the steviol xylosylated steviol glycoside of Formula II. For example, the yeast may be able to produce two, three, four, five, six, seven, eight, nine, ten, or more than ten different steviol glycoside species, with one or more of them being a xylosylated steviol glycoside.

The term “medium” refers to a liquid composition in which the engineered cell can be maintained, can grow, can ferment, or combinations thereof A “medium” may also be referred to as a “broth” or “cell culture,” and terms such as “growth,” “division,” “respiration,” and “fermentation” may be used to more specifically define the type of cellular activity that is occurring in the medium. “Cell culture” refers to the process of growing cells under controlled conditions, including the growth of prokaryotic and eukaryotic cells in one or more of a defined medium, a defined period of time, and defined temperature. Cell culture includes

A medium can be defined with regards to the components present in the medium, and amounts thereof, such as carbon sources, including (a) carbohydrates such as glucose and starch products such as maltodextrin; (b) nitrogen sources, such as yeast nitrogen base, ammonium hydroxide, urea, ammonium sulfate, or any combination thereof (c) salts, such as potassium phosphate (monobasic, dibasic), magnesium sulfate, sodium chloride, and calcium chloride; (d) vitamins, such as biotin, calcium pantothenate, folic acid, (myo)-inositol, nicotinic acid, p-aminobenzoic acid, pyridoxine HCl, riboflavin, thiamine HCL, and citric acid; (e) trace metals such as boric acid, copper sulfate, cobalt chloride, calcium chloride, potassium iodide, ferric chloride, magnesium sulfate, manganese chloride, sodium molybdate, and zinc sulfate. Components in the medium can be defined on a dry weight basis. Further, the medium is water-based, or an “aqueous” composition. The medium can also be defined with regards to its pH, and biocompatible acids, bases, and buffers that are used to control the pH in the medium.

A composition (a “feed composition”) can be added to the medium that includes the engineered cell to increase the volume of the medium, and as the engineered cell grows in the medium, the amount of biomass.

In some modes of practice, cell culture can be carried out in medium that includes steviol-containing compounds. In particular, the medium can include one of more steviol glycoside compounds of Formula I. Such compounds can be directly used by the glucosyltransferases polypeptide, such as one having at least 50% identity to SEQ ID NO:1, wherein the polypeptide is capable of transferring xylose from the activated xylose to the compound of Formula Ito form a xylosylated steviol glycoside compound of Formula II. Other sugars, such as rhamnose, galactose, arabinose, and/or glucose, may also be transferred to the compound of Formula I. In these aspects, the engineered cell is not required to have pathways to a steviol glycoside precursor (e.g., MVA, MEP, or SG pathways, such as described herein).

Exemplary engineered cells include those that are engineered yeast, bacteria, and fungus.

The “total steviol glycosides” refers all the steviol glycosides present in the medium after a period of cell culture, which includes the amount of steviol glycosides, including xylosylated steviol glycosides of the present disclosure, in the liquid medium and obtainable from the engineered yeast. The steviol glycoside content can be expressed with regards to a total steviol glycosides amount in the medium, or the amount of one or more, but not all, steviol glycosides, in the medium. The amount of steviol glycosides in the composition, including xylosylated steviol glycosides, can be expressed in relation to one another, or to the total amount of steviol glycosides, such as by a weight percentage of the total amount of steviol glycosides, or a ratio, or range of ratios, expressed as weight percent, or molar percent.

For recovery of xylosylated steviol glycosides, the medium can then be centrifuged or filtered to remove the engineered cells. The medium can optionally be treated to remove low molecular weight components (glucose, basic nutrients, and salts), such as by membrane dialysis. Depending on a desired use, a composition comprising one or more steviol glycoside compound(s) can be used.

If it is desired to provide a composition with xylosylated steviol glycosides in enriched or purified form, or where certain steviol glycosides are separated from one another, further purification can be carried out. Such enrichment or purification of steviol glycoside components can be carried out on the medium in which fermentation took place, or the medium can then be dried down prior to purification. For example, medium can be dried down using lyophilization to form a dry composition (e.g., powder or flakes) including steviol glycosides that can be subsequently processed.

As used herein, the term “total steviol glycosides” (TSG) is calculated as the sum of the content of all steviol glycosides in a composition on a dry (anhydrous) basis.

In some modes of practice, dried fermentation broth enriched for steviol glycosides is used as the starting material for purification. For example, a solvent or solvent combination can be added to the dried fermentation broth to dissolve or suspend material that includes the steviol glycosides. An exemplary combination for dissolving the steviol glycosides is a mixture of water and an alcohol (e.g., 50:50 ethanol:water). To facilitate dissolving or suspending, the dried broth materials can be heated at a temperature above room temperature, such as in the range of 40° C.-60° C. Mechanical disruption of the dried broth materials can also be performed, such as by sonication. The dissolved or suspended broth materials can be filtered using a micron or sub-micron prior to further purification, such as by preparative chromatography.

Dried fermentation broth enriched for steviol glycoside compounds can be subjected to purification or refinement, using methods such as crystallization or by using reverse phase liquid chromatography. Art-known techniques for enrichment and purification of steviol glycoside compounds include extraction using different solvents, adsorption and ion exchange chromatography, supercritical fluid extraction, crystallization, and ultra and nano membrane filtration (e.g., see Kumari, N. et al. (2017) Indian J Pharm Sci;79:617-624; Zhang, S. Q., et al. (2000) Food Res Int;33:617-20; Pol, J., et al. (2007) Anal Bioanal Chem; 388:1847-57; Puri, M., et al. (2012) Food Chem;132:1113-20; Teo, C. C., et al , Tan, S. N., et al. (2010) J Chromatogr A;121:2484-91). U.S. Pat. No. 5,962,678 discloses the re-crystallization of rebaudioside A using an anhydrous methanol solution to obtain an 80% pure rebaudioside A. U.S. Patent Publication No. 2006/0083838 discloses purification of rebaudioside A through re-crystallization with a solvent comprising ethanol and between 4 and 15% water. Japanese Patent Application No. 55-23756 discloses a method for purifying rebaudioside A and stevioside by crystallization from aqueous ethanol (>70%) to obtain an 80% pure rebaudioside A. U.S. Patent Publication No. 2007/0082103 discloses a method for purifying rebaudioside A by recrystallization from aqueous ethanol, asserting a two-step recrystallization from crude rebaudioside (60%) results in the formation of >98% pure rebaudioside at 97% yield. WO2007/149672 and WO2011/082288 disclose single step crystallization methods using organic solvents.

If chromatography is used, a suitable resin can be used to retain steviol glycoside compounds in the column, with removal of hydrophilic compounds which get washed through the column with a liquid such as water. Elution of steviol glycosides from the column can be accomplished a suitable solvent or solvent combination such as acetonitrile or methanol.

For example, steviol glycoside compounds can be purified using with preparative liquid chromatography, such as high pressure liquid chromatography (HPLC) or ultra-high pressure liquid chromatography (UHPLC). A steviol glycoside composition with xylosylated steviol glycoside can be dissolved in a mobile phase, such as a mixture of water and an alcohol (e.g., methanol) at a desired ratio (e.g., 60% water, 40% methanol, v/v). The composition can also be heated to enhance dissolution of the steviol glycoside material, such as heating at about 50° C. The solution can also be filtered prior to injection into the column, such as using a 0.2 μm filter. Phenomenex Kinetex XB-C18 5 μm, core-shell silica solid support, and stationary phase of C18 with iso-butyl side chains and TMS endcapping. The flow rate through the column can be based on column properties (such as about 20 mL/min), with a maximum pressure of 400 bar. Various xylosylated steviol glycosides can be identified by their elution times from the column. One of skill in the art will appreciate that the elution times for the xylosylated steviol glycosides can vary with changes in solvent and/or equipment.

Elution of xylosylated steviol glycosides from a reverse phase column can yield a composition which can be useful for any one of a variety of purposes. For example, a purified xylosylated steviol glycoside composition can be used as a sweetener composition for oral ingestion or oral use. The composition can be defined with regards to the steviol glycosides in the composition.

Sweetener compositions (also referred to as sweetening compositions), as used herein, refers to compositions that include one or more xylosylated steviol glycoside(s) of Formula II. In a preferred aspect the sweetener composition includes Compound E (SG-[13-β-Glu[(3→1)β-Glu](2→1)β-Glu(1-2)]-[19-β-Glu(2→1)β-Xyl]). For example, a sweetener composition can include one or more steviol glycoside(s) of Formula II (Formula II SGs) along with another steviol glycoside(s) that are outside of Formula II (non-Formula II SGs). If multiple steviol glycosides are present in the sweetener compositions, in some aspects non-Formula II SVs can be present in minor amounts in the composition (e.g., less than about 25%, less than about 20%, less than about 15%, or less than about 10%), of the total amount of steviol glycosides in the composition. One or more Formula II SGs can be present in a major amount in the composition, such as greater than about 75%, greater than about 80%, greater than about 85%, greater than about 90%, or greater than about 95%, of the total amount of steviol glycosides in the composition. In some aspects, Compound E (RebDG; SG-[13-β-Glu[(3→1)β-Glu](2→1)β-Glu(1-2)]-[19PβGlu(2→1)(3-Xyl]) is the primary SG of the SGs in the sweetener composition, meaning that it is present in an amount greater than any other SG in the composition. In some aspects, Compound E is present in an amount greater than the combined amount of all other SGs in the composition, i.e., Compound E is present in an amount of greater than 50% (wt) of the total SGs in the sweetener composition, and in some aspects greater than 60%, greater than 70%, greater than 80%, greater than 90%, greater than 95%, greater than 98%, or greater than 99% (wt), of the total amount of steviol glycoside in the composition.

The sweetener composition can optionally include another sweetener, an additive, a liquid carrier, or combinations thereof. Sweetener compositions are used to sweeten other compositions (sweetenable compositions) such as foods, beverages, medicines, oral hygiene compositions, nutraceuticals, and the like.

Sweetenable compositions, as used herein, mean substances which are contacted with the mouth of man or animal, including substances which are taken into but subsequently ejected from the mouth (such as a mouthwash rinse) and substances which are drunk, eaten, swallowed or otherwise ingested, and are suitable for human or animal consumption when used in a generally acceptable range. Sweetenable compositions are precursor compositions to sweetened compositions and are converted to sweetened compositions by combining the sweetenable compositions with at least one sweetening composition that include one or more xylosylated steviol glycoside(s) of Formula II. For example, a beverage with no sweetener component is a type of sweetenable composition. A sweetener composition including one or more xylosylated steviol glycoside(s) of Formula II, can be added to the un-sweetened beverage, thereby providing a sweetened beverage. The sweetened beverage is a type of sweetened composition.

In some preparations, one or more xylosylated steviol glycoside(s) of Formula II provide the sole sweetener component in a sweetening composition.

In some aspects, a sweetening composition includes one or more xylosylated steviol glycoside(s) of Formula II in an amount effective to provide a sweetness strength equivalent to a specified amount of sucrose. The amount of sucrose in a reference solution may be described in degrees Brix (°Bx). One degree Brix is 1 gram of sucrose in 100 grams of solution and represents the strength of the solution as percentage by weight (% w/w). For example, a sweetener composition contains one or more xylosylated steviol glycoside(s) of Formula II in an amount effective to provide a sweetness equivalent from about 0.50 to 14 degrees Brix of sugar when present in a sweetened composition, such as, for example, from about 5 to about 11 degrees Brix, from about 4 to about 7 degrees Brix, or about 5 degrees Brix.

The amount of xylosylated steviol glycoside(s) of Formula II in the sweetener composition may vary. One or more xylosylated steviol glycoside(s) of Formula II, can be present in a sweetener composition in any amount to impart the desired sweetness when the sweetener composition is incorporated into a sweetened composition. For example, one or more xylosylated steviol glycoside(s) of Formula II, are present in the sweetener composition in an amount effective to provide total steviol glycoside concentration from about 1 ppm to about 10,000 ppm when present in a sweetened composition, In another aspect, the one or more xylosylated steviol glycoside(s) of Formula II are present in the sweetener composition in an amount effective to provide a steviol glycoside concentration in the range of about 10 ppm to about 2,500 ppm, more specifically about 10 ppm to about 2000 ppm, about 10 ppm to about 1500 ppm, about 10 ppm to about 1250 ppm, about 10 ppm to about 1000 ppm, about 10 ppm to about 800 ppm, about 50 ppm to about 800 ppm, about 50 ppm to about 600 ppm, or about 200 ppm to about 500 ppm. Unless otherwise expressly stated, ppm is on a weight basis.

Optionally, a sweetener composition can also contain one or more additional non-steviol glycoside sweetener compound(s), such as a natural sweetener such as sucrose, fructose, glucose, erythritol, etc., or one or more synthetic sweeteners such as sucralose, potassium acesulfame, acesulfame acid and salts thereof, aspartame, saccharin and salts thereof, etc.

In addition to the one or more xylosylated steviol glycoside(s) of Formula II the sweetener compositions can optionally include a liquid carrier, binder matrix, additional additives, and/or the like. In some aspects, the sweetener composition contains additives including, but not limited to, carbohydrates, polyols, amino acids and their corresponding salts, poly- amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, weighing agents, gums, antioxidants, colorants, flavonoids, alcohols, polymers and combinations thereof. In some aspects, the additives act to improve the temporal and flavor profile of the sweetener to provide a sweetener composition with a favorable taste, such as a taste similar to sucrose.

The sweetener composition can also contain one or more functional ingredients, which provide a real or perceived heath benefit to the composition. Functional ingredients include, but are not limited to, saponins, antioxidants, dietary fiber sources, fatty acids, vitamins, glucosamine, minerals, preservatives, hydration agents, probiotics, prebiotics, weight management agents, osteoporosis management agents, phytoestrogens, long chain primary aliphatic saturated alcohols, phytosterols and combinations thereof.

Generally, the amount of functional ingredient in the sweetener composition or sweetened composition varies widely depending on the particular sweetener composition or sweetened composition and the desired functional ingredient. Those of ordinary skill in the art will readily ascertain the appropriate amount of functional ingredient for each sweetener composition or sweetened composition.

One or more xylosylated steviol glycoside(s) of Formula II, or sweetener compositions comprising these steviol glycosides, can be incorporated in any known edible material (referred to herein as a “sweetenable composition”) or other composition intended to be ingested and/or contacted with the mouth of a human or animal, such as, for example, pharmaceutical compositions, edible gel mixes and compositions, dental and oral hygiene compositions, foodstuffs (confections, condiments, chewing gum, cereal compositions, baked goods, baking goods, cooking adjuvants, dairy products, and tabletop sweetener compositions), beverages, and other beverage products (e.g., beverage mixes, beverage concentrates, etc.).

In one aspect, the sweetened composition is a beverage product comprising one or more xylosylated steviol glycoside(s) of Formula II. As used herein a “beverage product” is a ready-to-drink beverage, a beverage concentrate, a beverage syrup, frozen beverage, or a powdered beverage. Suitable ready-to-drink beverages include carbonated and non-carbonated beverages. Carbonated beverages include, but are not limited to, enhanced sparkling beverages, cola, lemon-lime flavored sparkling beverage, orange flavored sparkling beverage, grape flavored sparkling beverage, strawberry flavored sparkling beverage, pineapple flavored sparkling beverage, ginger- ale, soft drinks and root beer. Non-carbonated beverages include, but are not limited to fruit juice, fruit-flavored juice, juice drinks, nectars, vegetable juice, vegetable-flavored juice, sports drinks, energy drinks, enhanced water drinks, enhanced water with vitamins, near water drinks (e.g., water with natural or synthetic flavorants), coconut water, tea type drinks (e.g., black tea, green tea, red tea, oolong tea), coffee, cocoa drink, beverage containing milk components (e.g., milk beverages, coffee containing milk components, cafe au lait, milk tea, fruit milk beverages), beverages containing cereal extracts, smoothies and combinations thereof.

Examples of frozen beverages include, but are not limited to, icees, frozen cocktails, daiquiris, pina coladas, margaritas, milk shakes, frozen coffees, frozen lemonades, granitas, and slushees.

Beverage concentrates and beverage syrups can be prepared with an initial volume of liquid matrix (e.g., water) and the desired beverage ingredients. Full strength beverages are then prepared by adding further volumes of water. Powdered beverages are prepared by dry-mixing all of the beverage ingredients in the absence of a liquid matrix. Full strength beverages are then prepared by adding the full volume of water.

In one aspect, a beverage contains a sweetener composition comprising steviol glycosides, including one or more xylosylated steviol glycoside(s) of Formula II. Any sweetener composition comprising steviol glycosides, including one or more xylosylated steviol glycoside(s) of Formula II detailed herein can be used in the beverages. In another aspect, a method of preparing a beverage comprises combining a liquid matrix and steviol glycosides, including one or more xylosylated steviol glycoside(s) of Formula II. The method can further comprise addition of one or more sweeteners, additives and/or functional ingredients. In still another aspect, a method of preparing a beverage comprises combining a liquid matrix and a sweetener composition comprising steviol glycosides, including one or more xylosylated steviol glycoside(s) of Formula II.

In another aspect, a beverage contains a sweetener composition containing one or more xylosylated steviol glycoside(s) of Formula II, wherein the steviol glycosides are present in the beverage in an amount ranging from about 1 ppm to about 10,000 ppm, such as, for example, from about 25 ppm to about 800 ppm. In another aspect, steviol glycosides are present in the beverage in an amount ranging from about 100 ppm to about 600 ppm. In yet other aspects, steviol glycosides are present in the beverage in an amount ranging from about 100 to about 200 ppm, from about 100 ppm to about 300 ppm, from about 100 ppm to about 400 ppm, or from about 100 ppm to about 500 ppm. In still another aspect, steviol glycosides are present in the beverage in an amount ranging from about 300 to about 700 ppm, such as, for example, from about 400 ppm to about 600 ppm. In a particular aspect, steviol glycosides are present in the beverage in an amount of about 500 ppm.

In another aspect, granulated forms of one or more xylosylated steviol glycoside(s) of Formula II are provided. As used herein, the terms “granules,” “granulated forms,” and “granular forms” are synonymous and refer to free-flowing, substantially non-dusty, mechanically strong agglomerates of the steviol glycoside sweetener composition. Methods of granulation are known to those of ordinary skill in the art and are described in more detail in the PCT Publication WO 01/60842.

### Preparation of UGT Eenzymes

The amino acid sequence of SEQ ID NO:1 was fused to the C-terminal end of a GST protein and the sequence was codon optimized for E. coli expression. (DNA2.0, Menlo Park, CA). The resulting sequence was cloned into a proprietary expression vector (DNA2.0), which contains an IPTG inducible T5 promoter and strong RBS. The plasmid was the transformed into E. coli. The appropriate plasmid from Table 1 was transformed into BL21 cells using standard methods. Overnight cultures were grown in 250 mL flasks with 50 mL of LB plus 50 mg/mL Kanamycin at 30° C. and 250 RPM. The following morning the seed culture had an OD600=2.4 +/−0.5. The next morning, 3 liter Fernbach flasks containing either LB or Terrific Broth plus 50 mg/ml Kanamycin were inoculated with 16 ml of overnight culture, targeting an initial OD600=0.04. Cultures were grown for 5-7 hours at 250 RPM at the temperature shown in Table 1 and then induced with 0.8 mM IPTG. Following induction, cultures were incubated overnight at 250 RPM at the temperature shown in Table 1. Biomass was pelleted in 1 liter centrifuge bottles at 8000 RPM for 15 minutes at 4° C. Cells were resuspended in lysis buffer and lysed by sonication. Sonicate the cell suspension for 2 minutes with 20 second pulses followed 20 second rests on ice. The cells were transferred to 30 mL Oakridge tube and centrifuge at 15,000 RPM for 15 minutes. The supernatant was decanted into a new conical tube.

### Example 1: 19C Xylosylation of Stevioside, Reb A, Reb C, and Reb D Using Enzyme of SEQ ID NO:1 and UDP-Xylose

Six steviolglycoside (SG) ‘acceptors’ Reb A, B, C, D, M and stevioside were tested using 2 glucosyltransferase enzymes (SEQ ID NO:1 and UGT76G1) known to play key roles in SG biosynthesis in stevia leaves and activated xylose (UDP-xylose).

UDP-xylose (98.9% purity) and UDP-rhamnose di-sodium salt (95% purity) were purchased from BOC Sciences. The activated sugar donors were prepared to 10 mM concentration with 0.05 M tris buffer and stored according to manufacturers' recommendations before use.

Enzyme of SEQ ID NO:1 and UGT76G1 enzyme were prepared in E. coli host cells as tagged proteins with expression induced by a chemical inducer. Following induction, the cells were incubated overnight and harvested. The cells were then lysed (either through sonication, mechanical disruption or chemical lysis) and the proteins purified through chromatography and stored frozen at −20C till use. Enzyme activities were confirmed using UDP-glucose in a preliminary test. Frozen enzyme solution was thawed right before use and returned to frozen storage after use. Previous work showed that repeat freeze-thaw had very little effect on enzyme activities.

SG materials were obtained from various sources with 90%+ purities. All SG materials were prepared at 20 mM with 90% DMSO and stored at ambient temperature. Final DMSO concentration in the reaction mixture was 4.5%. Previous work demonstrated that this level of DMSO did not have any effect on bioconversion.

Sucrose, previously shown to have a stabilizing effect on the bioconversion reaction, was prepared at 0.1 M with 0.05 M tris buffer.

Tris (tris(hydroxymethyl)aminomethane) buffer was prepared at 0.05 M, pH 7.3 and contained 3 mM each of MgCl₂ and MnCl₂ as co-factors.

Bioconversion reaction was carried out in a total of 100 uL sealed vials containing the Tris buffer, 0.1 μmoles of SG acceptor, 0.2 μmoles of activated sugar donor, 1 μmoles of sucrose and 2 ug protein of the enzymes. To each vial, buffer, sucrose, UDP-sugar donor, and SG acceptor were added in that order before finally the enzymes were added. For each SG donor, a control was included to contain the same amounts of sucrose and enzymes without UDP-sugar donors. After hand mixing, the vials were placed in a temperature-controlled shaker set at 30C and 100 rpm in rotating motion for 96 hours (4 days).

After 4 days of reaction, 0.9 mL of 80% acetonitrile was added to each vial and the resulting samples were analyzed in LC/MS. Xylose modification of the -19C(O)-β-Glc residue was confirmed.

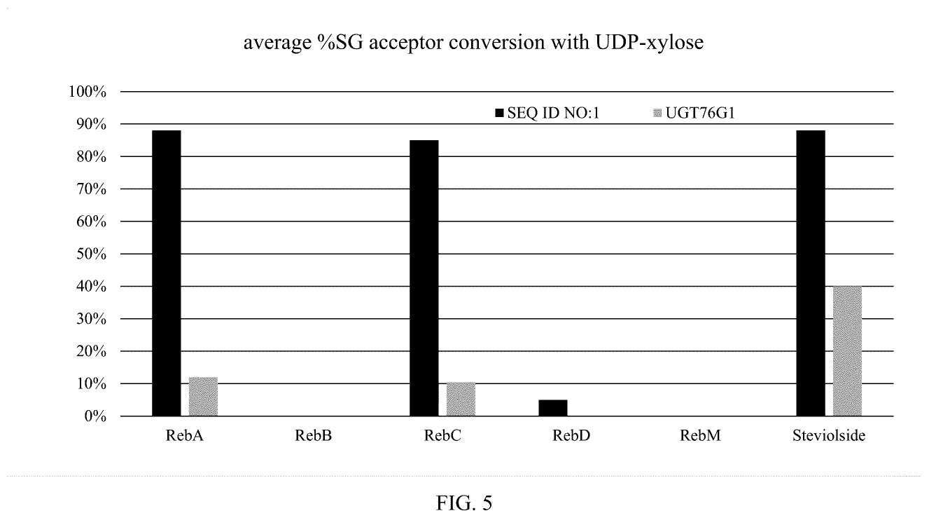

As shown in Table 1 and FIG. 5, polypeptide of SEQ ID NO:1 was more effective in transferring xylose to the SG acceptors than UGT76G1. This result was rather unexpected as UGT76G1 was postulated by others to have broader activities for acceptors and donors than SEQ ID NO:1. When polypeptide of SEQ ID NO:1 was employed, stevioside, Reb A and Reb C all showed very high conversion and one of the RebD replicate showed ˜10% conversion. When UGT76G1 was used, stevioside showed higher conversion than Reb A and Reb C.

TABLE 1
% SG bioconversion with UDP-xylose

| | | SG acceptor | | | | | |
|---|---|---|---|---|---|---|---|
| | | Reb | Reb | Reb | Reb | Reb | Stevi- |
| Enzyme | Run | A | B | C | D | M | oside |
| SEQ ID | 1 | 85.8% | 0% | 86.5% | 9.6% | 0% | 85.9% |
| NO: 1 | 2 | 90.2% | 0% | 84.0% | 0% | 0% | 90.2% |
| UGT76G1 | 1 | 12.4%* | 0% | 10.1% | 0% | 0% | 39.0% |
| | 2 | 11.2%* | 0% | 11.0% | 0% | 0% | 40.5% |

Note
*At the same enzyme dosage as the other SG acceptor, Reb A did not show any conversion. At 10x enzyme dose, ~12% Reb A was bioconverted.

### Example 2: Time Course Analysis of Glycosylation of Reb A Using SEQ ID NO:1 and Activated Sugars

The ability of SEQ ID NO:1 to glycosylate Reb A with different activated sugars used at different concentrations was examined, with the reaction products formed being measured over the course of the reaction.

Purified protein of SEQ ID NO:1 was used with UDP-glucose, UDP-xylose, and UDP-rhamnose di-sodium salts, and the reagents and reaction conditions were as described in Example 1.

TABLE 2

| | % Reb A bioconversion | | |
|---|---|---|---|
| Time, | UDP-Glucose | UDP-Xylose | UDP-Rhamnose |
| hours | (1 mM) | (1 mM) | (1 mM) |
| 0 | 0 | 0 | Not tested |
| 18 | 68.47 | 60.09 | Not tested |
| 48 | 68.6 | 60.39 | Not tested |
| 96 | 68.1 | 60.66 | Not tested |

TABLE 3

| | % Reb A bioconversion | | |
|---|---|---|---|
| Time, | UDP-Glucose | UDP-Xylose | UDP-Rhamnose |
| hours | (2 mM) | (2 mM) | (2 mM) |
| 0 | 0 | 0 | 0 |
| 18 | 84.93 | 83.26 | 14.7 |
| 48 | 83.9 | 83.5 | 25 |
| 96 | 82.1 | 82.33 | 30 |

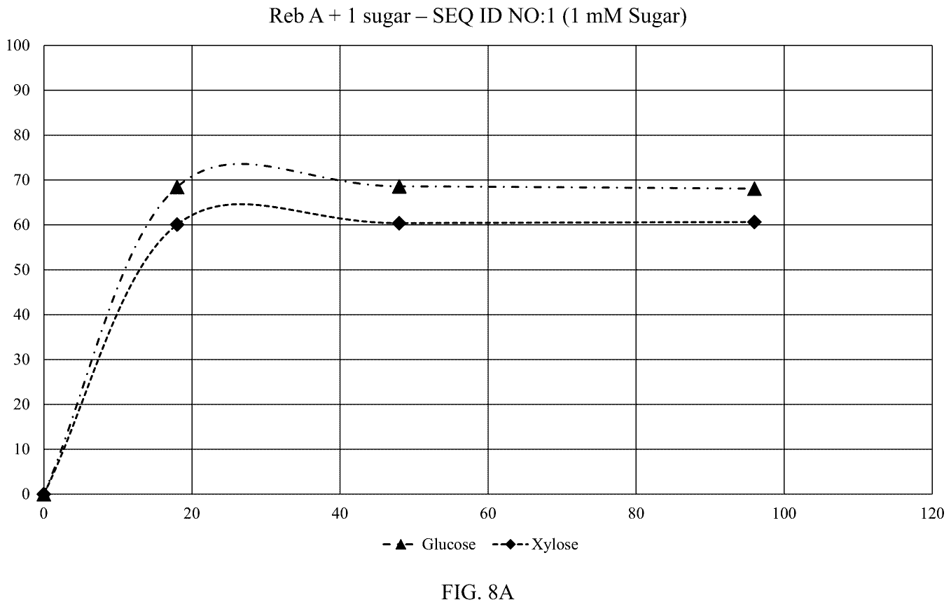

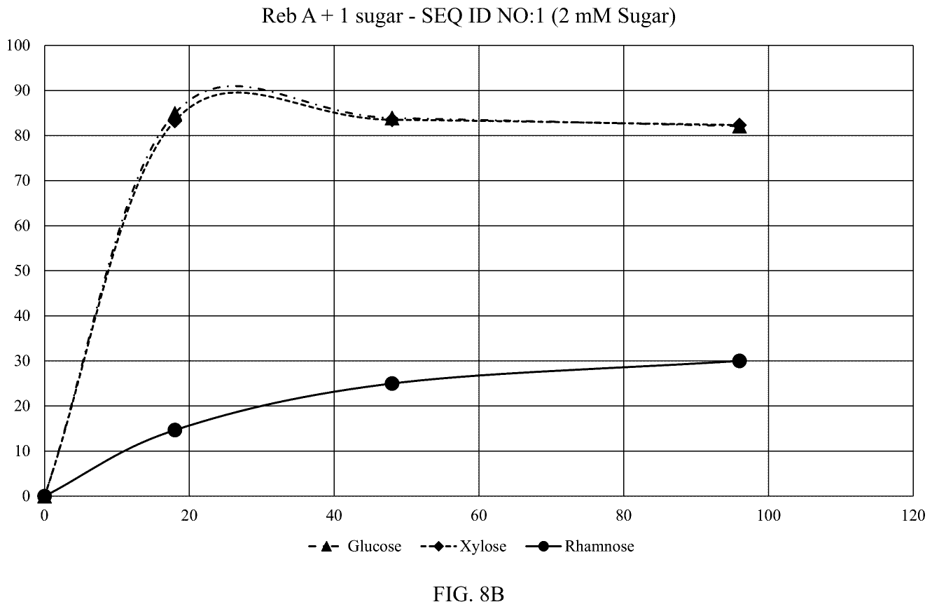

The polypeptide of SEQ ID NO:1 was able to effectively transfer glucose and xylose from UDP-glucose and USP-xylose, respectively, to RebA. In the presence of purified protein of SEQ ID NO:1 and 1 mM activated sugars, glucosylation and xylosylation of RebA plateaued after 20 minutes, with RebA having slightly higher levels of glucosylation than xylosylation (see Table 2 and FIG. 8A). In the presence of purified protein of SEQ ID NO:1 and 2 mM activated sugars, glucosylation and xylosylation of RebA plateaued after 20 minutes and were at similar levels, whereas rhamnosylation using UDP-rhamnose was significantly lower (see Table 3 and FIG. 8B).

### Example 3: Glycosylation of Reb F, Reb G, and Dulcoside A Using SEQ ID NO:1 and Activated Sugars

The ability of SEQ ID NO:1 to glycosylate the -19C(O)-β-Glc residue of RebF, RebG, and Dulcoside A with different activated sugars used at different concentrations was examined, with the reaction products formed being measured over the course of the reaction.

UDP-glucose, UDP-xylose, and UDP-rhamnose di-sodium salts were used, and the reagents and reaction conditions were as described in Example 1.

TABLE 4
(glycosylation of Reb F)

| Time, | % Reb F bioconversion | | |
|---|---|---|---|
| hours | UDP-Glucose | UDP-Xylose | UDP-Rhamnose |
| 0 | 0 | 0 | 0 |
| 15 | 85.9 | 86.406 | 17.5 |
| 48 | 85.96 | 86.2 | 28.412 |
| 96 | 84.4 | 84.179 | 33.216 |

TABLE 5
(glycosylation of Reb G)

| Time, | % Reb G bioconversion | | |
|---|---|---|---|
| hours | UDP-Glucose | UDP-Xylose | UDP-Rhamnose |
| 0 | 0 | 0 | 0 |
| 15 | 87.748 | 78.904 | 17.055 |
| 48 | 85.225 | 78.311 | 26.923 |
| 96 | 83.427 | 77.211 | 31.347 |

TABLE 6
(glycosylation of Dulcoside A)

| Time, | % Dul A bioconversion | | |
|---|---|---|---|
| hours | UDP-Glucose | UDP-Xylose | UDP-Rhamnose |
| 0 | 0 | 0 | 0 |
| 15 | 77.162 | 79.34 | 17.384 |
| 48 | 75.215 | 79.919 | 27.427 |
| 96 | 73.71 | 79.349 | 32.608 |

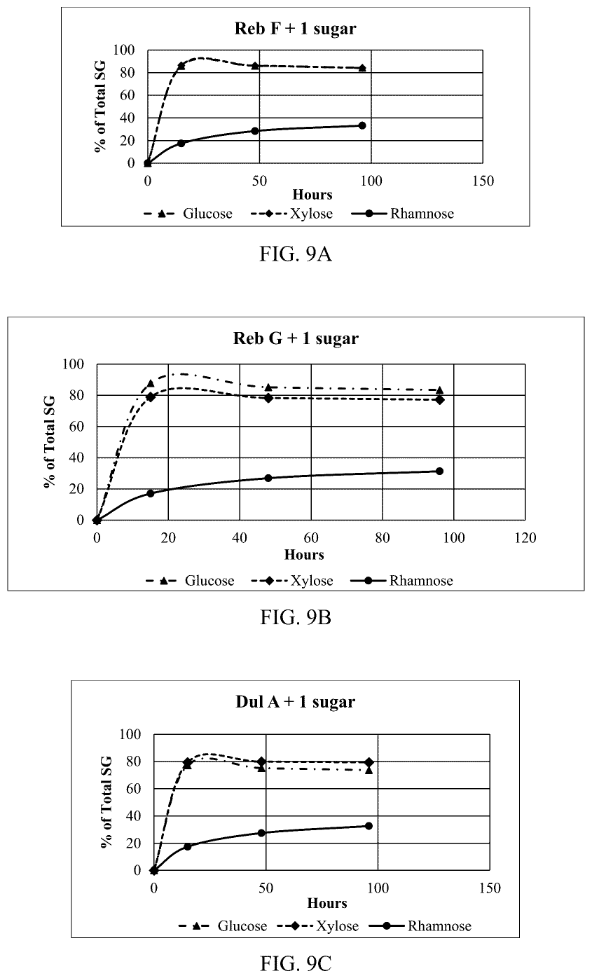

SEQ ID NO:1 was able to effectively transfer glucose and xylose from UDP-glucose and UDP-xylose, respectively, to the -19C(O)-β-Glc residue of Reb F, Reb G, and Dulcoside A, with glucosylation and xylosylation plateauing after 20 minutes. Rhamnosylation using UDP-rhamnose was significantly lower (see Tables 4-6 and FIGS. 9A-C).

### Example 4: Glycosylation of Reb A Using SEQ ID NO:1 Homologs and Variants

In vitro Enzyme Production

Candidate xylosyltransferase enzymes are produced in vitro using the PURExpress In Vitro Protein Synthesis kit from New England Biolabs (NEB #E6800) according to the manufacturer's instructions, including 250 ng plasmid DNA and the addition of 20 units RNase Inhibitor, Murine (NEB #M0314).

Glycosyltransferase assay for enzyme produced by In Vitro Protein Synthesis

The steviol glycoside (SG) ‘acceptors’ Reb A was tested using 4 glucosyltransferase enzymes (SEQ ID NOs: 1-4) homologous to glucosyltransferases in SG biosynthesis in stevia leaves. The identity of SEQ ID NOs: 2-4 to SEQ ID NO:1 is listed in Table 7.

TABLE 7

| | Identity to SEQ ID NO: 1 |
|---|---|
| SEQ ID NO: 1 | 100% |
| SEQ ID NO: 2 | 99% |
| SEQ ID NO: 3 | 27% |
| SEQ ID NO: 4 | 25% |

UDP-xylose (98.9% purity) was purchased from BOC Sciences. The activated sugar donors were prepared to 10 mM concentration with 0.05 M tris buffer and stored according to manufacturers' recommendations before use. SG materials were obtained from various sources with 90%+ purities. All SG materials were prepared at 20 mM with 90% DMSO and stored at ambient temperature. Final DMSO concentration in the reaction mixture was 4.5%. Previous work demonstrated that this level of DMSO did not have any effect on bioconversion.

Tris (tris(hydroxymethyl)aminomethane) buffer was prepared at 0.05 M, pH 7.3 and contained 3 mM each of MgCl₂ and MnCl₂ as co-factors.

Enzymes were produced via in vitro transcription-translation (IVTT) and activities were confirmed using UDP-glucose in a preliminary test. Bioconversion reaction was carried out in a total of 100 μL sealed vials containing the Tris buffer, 0.1 μmoles of SG acceptor, 0.2 μmoles of activated sugar donor, 1 μmoles of sucrose and 2 μg protein of the enzymes. To each vial, buffer, UDP-sugar donor, SG acceptor were added in that order before, finally, the enzymes were added. For each SG donor, a control was included to contain the same amounts of enzymes without UDP-sugar donors. After hand mixing, the vials were placed in a temperature-controlled shaker set at 30C and 100 rpm in rotating motion for 96 hours (4 days).

After 4 days of reaction, 0.9 mL of 80% acetonitrile was added to each vial and the resulting samples were analyzed in LC/MS. Xylose modification of the -19C(O)-β-Glc residue was confirmed.

Activity of SEQ ID NOs: 2-4 is expressed relative to the activity of SEQ ID NO:1. As shown in Table 8, SEQ ID NO:1 was more effective in transferring xylose to the SG acceptor than either SEQ ID NO: 3 or 4. The substantially overall increased activity of SEQ ID NO:2 relative to SEQ ID NO:1 was rather unexpected. In the presence of SEQ ID NO: 1 and 2 very high conversion of Reb A to the xylosylated produce occurred in the presence of both UDP-glucose and UDP-xylose. [SEQ ID NO:3 was run more than once in this experiment,]

TABLE 8

| | SEQ ID1 | SEQ ID2 | SEQ ID3 | SEQ ID3 | SEQ ID4 |
|---|---|---|---|---|---|
| | Glucose | Glucose | Glucose | Glucose | Glucose |
| 18 hrs | 100% | 825 | 81.5 | 148 | 174 |
| 48 hrs | 100% | 578 | 87.1 | 132 | 151 |
| 96 hrs | 100% | 504 | 99.1 | 148 | 170 |
| | Xylose | Xylose | Xylose | Xylose | Xylose |
| 18 hrs | 100% | 459 | 47.1 | 78.1 | 11.5 |
| 48 hrs | 100% | 392 | 51.0 | 78.6 | 9.68 |
| 96 hrs | 100% | 312 | 38.9 | 62.1 | 10.9 |

1. A method for forming a glycosylated steviol glycoside, the method comprising
forming a compound of Formula II from a compound of Formula I in vitro or in an engineered cell using a glycosyltransferase having UDP-xylose:19-steviol xylosyltransferase activity, wherein
Formula I is: 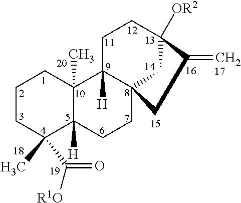
wherein R¹ comprises a glucose residue, and R² comprises one or more sugar residues or is a hydrogen, and (ii) an activated xylose;
the glycosyltransferase transfers xylose from an activated xylose to the compound of Formula I; and
Formula II is: 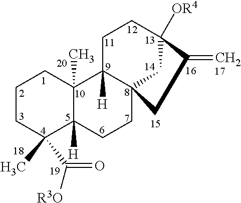
wherein R³ comprises one or more xylose residue(s) added by the glycosyltransferase, and R⁴ is the same as R² or comprises one or more additional sugar residue(s).

2. The method of claim 1, wherein R³ of Formula II is an oligosaccharide moiety comprising one or more xylose residue(s).

3. The method of claim 1, wherein the oligosaccharide moiety is -b-Glu-b-Xyl or -b-Glu-b-Xyl-R⁵, wherein R⁵ comprises one or more sugar residue(s).

4. The method of claim 3, wherein R³ of Formula II consists of -Glu-b-Xyl.

5. The method of claim 1 where the compound of Formula I is selected from the group consisting of stevioside, rebaudioside A, rebaudioside C, rebaudioside D, rebaudioside F, rebaudioside G, and dulcoside-A.

6. The method of claim 5 where the compound of Formula I is selected from the group consisting of stevioside, rebaudioside A, and rebaudioside C.

7. The method of claim 5 where the compound of Formula I is rebaudioside A.

8. The method of claim 3 wherein the compound of Formula II is:

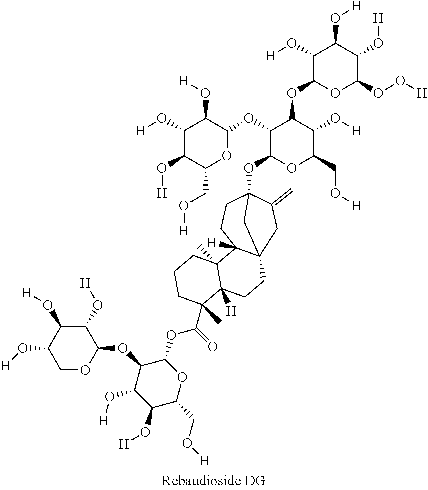

9. The method of claim 1 wherein the activated xylose is UDP-xylose.

10. The method of claim 1 wherein the glycosyltransferase has at least 75% sequence identity to SEQ ID NO:1.

11. The method of claim 10 wherein the glycosyltransferase 85% or greater sequence identity to SEQ ID NO:1.

12. The method of claim 1 wherein the glycosyltransferase comprises the following amino acids relative to SEQ ID NO:1:
(A) W22, L23, A24, F25, G26, H27, L28, L29, P30, L57, P59, D90, V91, P92, H93, D94, R95, P96, D97, M98, V99, D128, F130, L149, L150, G151, S152, M155, R190, M191, K192, R195, T196, K197, S199, S200, G201, M202, S203, L204, A205, R221, P249, P250, L251, Y277, A279, L280, G281, S282, E283, V284, P285, A308, L309, R310, R338, W339, V340, P341, Q342, M343, L346, F354, H357, C358, G359, W360, N361, S362, T363, E365, I378, F379, G380, D381, Q382, and N385;
(B) M1, H16, V18, P21, W22, L23, A24, F25, G26, H27, P30, L34, L38, A39, G42, H43, S46, S49, T50, P51, N53, R56, L57, P58, V71, P76, L81, P82, A85, E86, T88, D90, A105, D107, L109, L117, D123, D128, W133, A138, A153, P180, E187, S200, R207, R221, S222, E225, E227, P241, G246, P249, W266, L267, Q270, S274, V275, Y277, V278, A279, G281, S282, E283, E293, L294, A295, G297, L298, E299, F305, W307, R310, L321, P322, G324, F325, R328, G333, V335, W339, P341, Q342, I345, L346, H348, V351, G352, F354, L355, T356, H357, G359, S362, E365, L373, L376, P377, D381, Q382, G383, N385, A386, R387, G395, V398, R400, D404, G405, F407, V412, A413, V419, and A433; or
both (A) and (B)

13. The method of claim 1 wherein the glycosyltransferase comprises one or more of the following amino acids motifs relative to SEQ ID NO:1: WLAFGHLLP (SEQ ID NO:5), LPP; NDVPHDRPDMV (SEQ ID NO:6), DVF, LLGSAHM (SEQ ID NO:7); RMKLIRTKGSSGMSLA (SEQ ID NO:8); PPL; YVALGSEVP (SEQ ID NO:9); ALR; RWVPQMSIL (SEQ ID NO:10); FLTHCGWNSTIE (SEQ ID NO:11); IFGDQGPN (SEQ ID NO:12).

14. The method of claim 1 which is performed in vitro using a steviol glycoside reaction composition comprising (i) the steviol glycoside compound of Formula I, (ii) the activated xylose sugar; and (iii) the glycosyltransferase having UDP-xylose: 19-steviol xylosyltransferase activity.

15. The method of claim 14 where the compound of Formula I constitutes more than 50% (mol) of steviol glycoside in the reaction composition.

16. The method of claim 14 where, in the reaction composition, more than 10%, more than 20%, more than 30%, more than 40%, more than 50%, more than 60%, more than 65%, more than 75%, or more than 80% of the steviol glycoside compound of Formula I is converted to the xylosylated steviol glycoside compound of Formula II.

17. The method of claim 16 wherein the compound of Formula II is Rebaudioside DG.

18. The method of claim 14 where the reaction composition comprises one or more of the following: (a) a magnesium and/or manganese salt; (b) a pH in the range of 3-9, 4-8, 6.8-7.8, or 7.1-7.5; (c) a molar excess of UDP-xylose (UDP-X) to the steviol glycoside compound of Formula I (SGI), or a UDP-X: SGI molar ratio in the range of 1:1 to 1:100, 1:5 to 1:10, 1:20 to 1:50, 1:50-1:100, or 1.1:1 to 10:1, 1.2:1 to 5:1, or 1.5:1 to 3:1, where the activated xylose comprises the UDP-xylose (UDP-X); (d) a polypeptide (wt) to UDP-X (mol) ratio in the range of 50 mg: 1 mmol to 10 mg: 1 mmol, in the range of 25 mg: 1 mmol to 15 mg: 1 mmol, or about 20 mg: 1 mmol; (e) a polypeptide (wt) to SGI (mol) ratio in the range of 25 mg: 1 mmol to 5 mg: 1 mmol, in the range of 15 mg: 1 mmol to 7.5 mg: 1 mmol, or about 10 mg: 1 mmol.

19. The method of claim 14, wherein the reaction comprises one or more of the following conditions: (a) a time in the range of 1 hour to 10 days, in the range of 1 hour to 24 hours, in the range of 1 hour to 12 hours, in the range of 12 hours to 7 days, or in the range of 1 day to 5 days; (b) at a temperature in the range of 5-95° C., 25-80° C., 25-40° C., 30-40° C., 40-50° C., 50-60° C., 60-70° C., 70-80° C., or 25-35° C., or 28-32° C.

20. The method of claim 14, further comprising one or more steps of separating the glycosylated steviol glycoside compound of Formula II from one or more other components in the composition.

21. The method of claim 1,
wherein forming is performed in an engineered cell capable of making the steviol glycoside compound of Formula I; and
wherein the activated xylose is fed (externally) to the cell, or the engineered cell is capable of making the activated xylose, and xylose is transferred from the activated to the compound of Formula Ito form the xylosylated steviol glycoside compound of Formula II.

22. An engineered cell comprising
a pathway for forming a steviol glycoside compound of Formula I: 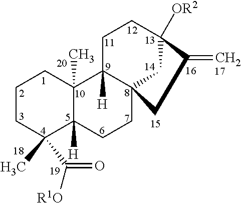
wherein R¹ comprises a glucose residue, and R² comprises one or more sugar residues or is hydrogen,
wherein the pathway provides the steviol glycoside compound of Formula I in an amount that is greater than an amount of steviol glycoside not of formula I that may be formed in the cell; and
a glycosyltransferase having UDP-xylose:19-steviol xylosyltransferase activity, wherein the glycosyltransferase is capable of transferring one or more of xylose residues to the compound of Formula I to form a glycosylated steviol glycoside compound of Formula II: 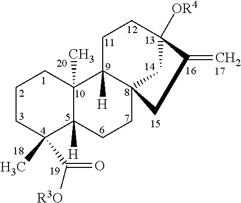
wherein R³ of Formula II comprises one or more xylose residue(s), and wherein R⁴ is the same as R² or includes one or more additional sugar residue(s).

23. The engineered cell of claim 22, wherein the engineered cell comprises a pathway for the production of UDP-xylose.

24. The engineered cell of claim 23, where, in the engineered cell, the pathway for the production of UDP-xylose comprises UDP-glucose dehydrogenase, UDP-glucuronic acid decarboxylase, UDP-xylose synthase, or a combination thereof.

25. The engineered cell of claim 22, where the engineered cell comprises one or more UDP-glucose recycling enzyme(s) under heterologous gene control and/or one or more UGT(s).

26. (canceled)

27. The engineered cell of claim 22 wherein the engineered cell is an engineered yeast selected from the group consisting of species of Candida, Kloeckera (Hanseniaspora), Issatchenkia, Kluyveromyces, Lipomyces, Pichia (Hansenula), Rhodotorula, Saccharomycete, Saccharomyces, Schizosaccharomyces, Torulopsis, Torulaspora, Yarrowia, and Zygosaccharomyces.

28. The engineered cell of claim 22 wherein the engineered cell is an engineered bacterial cell selected from the group consisting of species of Escherichia and Bacillus.

29. The engineered cell of claim 22 wherein the engineered cell expresses one or more exogenous nucleic acid(s) encoding one or more of the following proteins heterologous to the cell: GGPPS polypeptide, an ent-copalyl diphosphate synthase (CDPS) polypeptide, a kaurene oxidase (KO) polypeptide, a kaurene synthase (KS) polypeptide; a steviol synthase (KAH) polypeptide, a cytochrome P450 reductase (CPR) polypeptide, a UGT74G1 polypeptide, a UGT76G1 polypeptide.

30. A method for forming a xylosylated steviol glycoside, the method comprising
forming a xylosylated steviol glycoside product from a steviol glycoside reactant in a reaction composition comprising (i) one or more of stevioside, rebaudioside A, rebaudioside C, rebaudioside F, rebaudioside G, or dulcoside A; (ii) UDP-xylose; (iii) a polypeptide of SEQ ID NO:1 or SEQ ID NO:2, (iv) a non-activated sugar, and (v) a magnesium and/or manganese salt;
wherein the reaction composition has a pH in the range of 4.8-7.8;
wherein the one or more of stevioside, rebaudioside A, rebaudioside C, rebaudioside F, rebaudioside G, or dulcoside A are present at a total of 80% (mol) or greater of all of steviol glycoside in the reaction composition;
wherein the polypeptide (wt) to the one or more of stevioside, rebaudioside A, rebaudioside C, rebaudioside F, rebaudioside G, or dulcoside A (mol) ratio is in the range of 15 mg: 1 mmol to 7.5 mg: 1 mmol;
reacting the reaction composition for at least 12 hours at a temperature in the range of 25-95° C., to provide a product composition comprising one or more of Compounds D, E, G, H, I, or J:

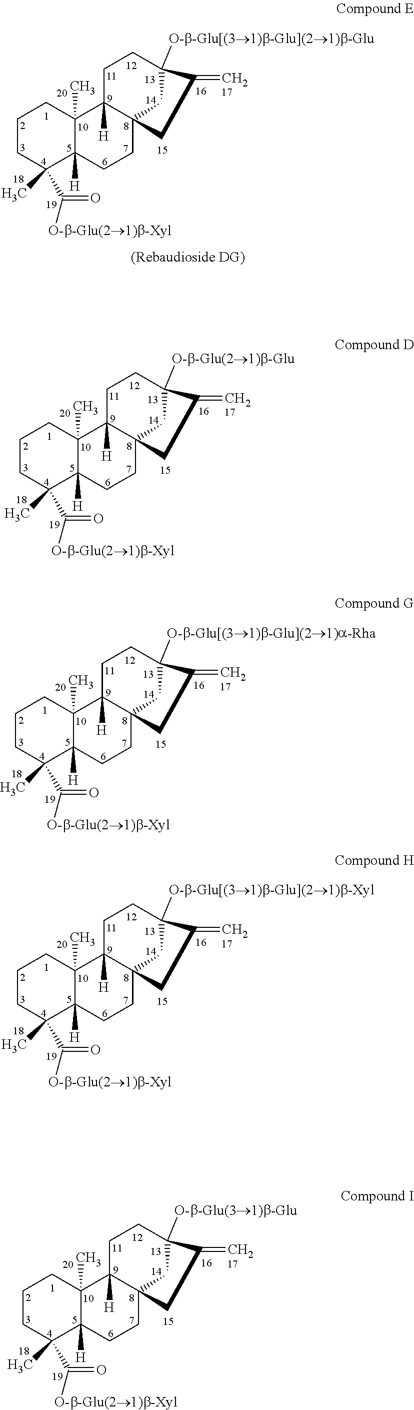

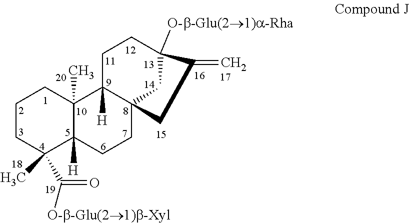

31. A composition comprising the xylosylated steviol glycoside compound formed by the method of claim 1.

32-37. (canceled)

38. A composition comprising the xylosylated steviol glycoside compound formed by the method of claim 30.
